(19) United States
(12) Patent Application Publication
(10) Pub. No.: US 2011/0256100 A1
(43) Pub. Date: Oct. 20, 2011

(54) SECRETED MODULAR CALCIUM BINDING PROTEIN FOR INTRACELLULAR MODULATION OF BONE MORPHOGENETIC PROTEIN SIGNALING

(75) Inventors: John Terrig Thomas, Arlington, VA (US); Malcolm C. Moos Jr., Bethesda, MD (US); Frank Luyten, Kraainem (BE)

(73) Assignee: KATHOLIEKE UNIVERSITEIT LEUVEN, Leuven (BE)

(21) Appl. No.: 13/057,545

(22) PCT Filed: Jul. 31, 2009
(86) PCT No.: PCT/US2009/052373

§ 371 Date: Apr. 28, 2011

(51) Int. Cl. A61K 38/17; A61K 31/7088; A61P 19/08; A61P 19/00; A61P 19/02; A61P 19/04; A61K 38/16; A61K 35/00

(52) U.S. Cl. 424/93.1; 514/16.7; 514/44 R; 514/1.1; 514/8.8

(57) ABSTRACT

A method for modulating Bone Morphogenetic Protein (BMP) signaling activity in a cell or tissue of a vertebrate subject is provided which comprises administering a polynucleotide or polypeptide encoding a Secreted Modular Calcium Binding Protein (SMOC) polypeptide, or a conservatively modified variant, derivative, or analog thereof, in an amount effective to activate intracellular Mitogen Activated Protein (MAP) kinase activity and to reduce BMP signaling activity in the cell or tissue of the vertebrate subject. Methods for treating joint disorders in a mammalian subject are provided.

### CROSS REFERENCE TO RELATED APPLICATIONS

This application claims the benefit of U.S. provisional application Ser. No. 61/086,679, filed Aug. 6, 2008, which is incorporated herein by reference in its entirety.

### FIELD

The present invention generally relates to the field of cell growth, differentiation and control of formation of anatomic patterns, and particularly, skeletal development, in a vertebrate subject. Embodiments of the present invention provide methods for modulating bone morphogenetic protein (BMP) signaling activity in a cell or tissue of a vertebrate subject and methods for treating joint disorders in a mammalian subject by administering a secreted modular calcium binding protein (SMOC) polypeptide, or a conservatively modified variant, derivative, or analog thereof, or by administering a polynucleotide encoding a SMOC polypeptide, or a conservatively modified variant, derivative, or analog thereof, in an amount effective to activate intracellular mitogen activated protein (MAP) kinase activity and to reduce BMP signaling activity in the cell or tissue of the subject.

### BACKGROUND

Patterning of the body axis, axial and appendicular skeleton, and various other structures requires many interacting signals expressed in complex spatial and temporal patterns. Among these signals are the Bone Morphogenetic Proteins (BMPs) and their antagonists (for review, see Vonica et al., Semin Cell Dev Biol 17: 117-132, 2006). Several proteins in the BMP subgroup of the Transforming Growth Factor superfamily were identified by classical biochemical purification and protein sequencing of fractions containing potent bone forming activity from bovine cartilage (Chang et al., J Biol Chem 269: 28227-28234, 1994). These fractions also contained proteins unrelated to the BMPs structurally, such as the Wnt antagonist Frzb (Hoang et al., J Biol Chem 27: 26131-26137, 1996; Wang et al., Cell, 1997, 88, 757-766). Another protein, which could not be dissociated from osteoinductive activity following extensive purification, was identified as Secreted Modular Calcium-Binding Protein-2 (SMOC-2). SMOC-2 and the closely related SMOC-1 have been classified as belonging to the BM-40 family of modular extracellular proteins (Vanhamme et al., 2002; 2003) because they contain a follistatin-like (FS) domain and a C-terminal extracellular calcium-binding (EC) domain (Vanhamme et al., 2002; 2003). They also contain two thyroglobulin-like (TY) domains and a novel domain without known homologs. The EC domain has been shown to bind calcium (Vanhamme et al., 2002), but data regarding the biological function of SMOC1/2 remain limited. There are currently no published data on SMOC-1/2 expression or function during embryological development. However, these proteins are expressed in a wide variety of adult mouse tissues and are secreted by established cell lines of epithelial and mesenchymal origin. Immunofluorescence analyses have shown SMOC-1/2 to be associated with basement membrane structures (Vanhamme et al., 2002; 2003) and human vascular endothelial cells (HUVECs) infected with Adenovirus expressing SMOC-2 show SMOC-2 to be localized predominantly to the cell periphery (Rocnik et al., J Biol Chem 281: 22855-22864, 2006). These data are consistent with a putative role of SMOC-2 as a regulator of extracellular matrix interactions and/or growth factor signaling. The BM-40 family member Secreted Protein Acidic and Rich in Cysteine (SPARC) binds to platelet-derived growth factor (PDGF; Raines et al., Proc Natl Acad Sci USA 89: 1281-1285, 1992) and vascular endotheleial growth factor (VEGF; Kupprion et al., J Biol Chem 273: 29635-29640, 1998) and indirectly influences the effects of basic fibroblast growth factor (bFGF; Hasselarr and Sage, 1992) and transforming growth factor beta (TGF-β; Francki et al., J Biol Chem 274: 32145-32152, 1999). SMOC-2 has been shown to potentiate cellular responses to bFGF and VEGF (Rocnik et al., J Biol Chem 281: 22855-22864, 2006). Studies indicate that modulation of BMP signaling inhibits the onset and progression of joint disease, such as joint ankylosis. (Lories et al., J. Clin. Invest. 115: 1571-1579, 2005.)

A need exists in the art for improved therapy for joint disorders or joint diseases, such as spondylarthropathies, in a vertebrate subject. Therapeutic compositions are needed that modulate Bone Morphogenetic Protein (BMP) signaling activity in a cell or tissue of a vertebrate subject that can be used to treat disease, to modulate skeletal development, and to improve growth and differentiation of bone or cartilage in the vertebrate subject in need thereof.

### SUMMARY

Aspects of the present invention relate to methods for modulating cell growth and differentiation, including skeletal development, in a vertebrate subject, and further relate to methods for modulating growth and differentiation of bone and cartilage in the vertebrate subject. Methods for modulating bone morphogenetic protein (BMP) signaling activity in a cell or tissue of a vertebrate subject are provided that comprise administering to the subject a secreted modular calcium binding protein (SMOC) polypeptide, or a conservatively modified variant, derivative, or analog thereof, or administering a polynucleotide encoding a SMOC polypeptide, or a conservatively modified variant, derivative, or analog thereof, in an amount effective to activate intracellular mitogen activated protein (MAP) kinase activity and to reduce BMP signaling activity in the cell or tissue of the subject.

Particular embodiments of the invention provide methods for modulating bone morphogenetic protein activity that comprise activating an extracellular signal-regulated mitogen-activated protein kinase with a secreted modular calcium binding protein. Further embodiments of the invention relate to methods for modulating growth and differentiation of bone and cartilage in a patient that comprise administering to the patient an effective amount of a secreted modular calcium binding protein; a nucleic acid encoding a secreted modular calcium binding protein; a vector comprising a nucleic acid encoding a secreted modular calcium binding protein; or a host cell expressing a secreted modular calcium binding protein. Finally, other aspects of the invention are directed to methods for treating musculoskeletal disorders that comprise administering to a patient suffering from such a disorder a therapeutically effective amount of a secreted modular calcium binding protein; a nucleic acid encoding a secreted modular calcium binding protein; a vector comprising a nucleic acid encoding a secreted modular calcium binding protein; or a host cell expressing a secreted modular calcium binding protein.

### BRIEF DESCRIPTION OF THE DRAWINGS

FIG. 1 shows expression of XSMOC-1 during embryogenesis. (A) RT-PCR analysis of XSMOC-1 expression at different stages of development. XSMOC-1 was not detectable until after stage 12. Histone H4 is shown as positive control and -RT indicates RT-PCR without reverse transcriptase as negative control. (B-I) Whole mount hybridization in situ analysis of XSMOC-1 (anterior to the left). (B) Ventral view of a stage 12.5 embryo showing anterior staining (C) Ventro-lateral view of a stage 15 embryo showing anterior and lateral staining (D) Lateral, and (E) Dorsal view of a stage 17 embryo showing staining lateral to the neural plate. (F-H) Lateral views showing XSMOC-1 expression throughout the developing pronephros at Stage 20 (F), Stage 22 (G), Stage 24 (H). At stage 22 (G), additional expression was observed dorsal to the cement gland (arrowhead), and from stage 25 (H) onwards XSMOC-1 was expressed in the ventral region of the developing eye. (I, J) Dorsal views of (H), following prolonged color development, showed expression in the mesencephalon, rhombencephalon (white arrows) and migrating neural crest (black arrows). (K-M) Transverse sections taken through (I, J) in the region of the forebrain (K), hindbrain (L), and anterior trunk (M). Staining was prominent in the ventral aspect of the developing eye (K) and in the lateral regions of the hindbrain (L). Within the trunk (M), staining was observed in the pronephros and in subepitheleial migrating neural crest cells. E, eye, FB, forebrain, HB, hindbrain, N, notochord, NC, neural crest, NT neural tube, S, somite.

FIG. 2 shows that Xenopus embryos overexpressing XSMOC-1 exhibit a dorsalized phenotype. (A-D) Dorsal views of stage 17 embryos injected bilaterally at the two-cell stage with 300 pg GFP (A, C) or XSMOC-1 (B, D) mRNAs. XSMOC-1-injected embryos have exaggerated anterior and diminished posterior structures (B) with laterally expanded expression of the neural plate marker Sox2 (D) Arrows indicate the position of the neural tube. (E-F) Transverse sections taken through the anterior regions of overstained embryos C and D (white bars) show Sox2 expression throughout the dorsal tissues in XSMOC-1 injected embryos (F). The phenotypes shown for XSMOC-1 overexpression are typical for this stage and were observed in 95% of the embryos in three separate experiments (n=96).

FIG. 3 shows that dorsalization is pronounced in tadpoles overexpressing XSMOC-1. (A-F) Stage 26 Xenopus embryos injected bilaterally at the 2-cell stage with 300 pg GFP (A, C, E) or XSMOC-1 mRNAs (B, D, F). XSMOC-1 injected embryos were dorsalized, with exaggerated dorsal/anterior structures, particularly cement glands. The XSMOC-1 overexpression phenotypes shown in (B) were typical for this stage and were observed 95% of the embryos in five independent experiments (n=218). (C, D) Histological 3 mm plastic sections (modified Von Gieson stain) showing hypertrophic cement gland cells in XSMOC-1 overexpressing embryos (D). (E, F) 7 mm paraffin sections (Feulgen, light green, orange G) showing enlargement of the neural tube and disorganized somites in XSMOC-1 overexpressing embryos (F).

FIG. 4 shows that XSMOC-1 induces neural markers in animal cap explants and acts non-cell autonomously. (A) RT-PCR analysis of animal caps obtained from embryos injected bilaterally with 300 pg GFP (control) or XSMOC-1 at the 2-cell stage. Animal caps were removed from stage 8 embryos and cultured until non-injected siblings reached stage 17. XSMOC-1 induced the neural markers N-CAM, NRP1, Otx2, and XAG1 and suppressed the expression of the epidermal marker, keratin. mRNA extracted from whole embryos (lane 3) was used as a positive control for the RT-PCR reactions; reactions from which reverse transcriptase was omitted (-RT, lane 4) were the negative controls. (B, C) Whole mount hybridization in situ of Otx2 in albino animal caps conjugated to wild-type caps. Wild-type embryos were injected bilaterally with 300 pg GFP (B) or XSMOC1 (C). Animal caps were removed at stage 8 and conjugated to caps removed from stage 8 non-injected albino embryos. The conjugates were cultured until sibling embryos reached stage 17. Otx2 staining was not observed in the GFP control cap conjugates (B), but was present in the non-injected albino caps conjugated to XSMOC1-injected wild-type caps (C).

FIG. 5 shows that unilateral injection of XSMOC-1 antisense morpholino (MO) produces mild ventralization and anophthalmia on the injected side. XSMOC-1 MO (6 ng) was injected into a single blastomere at the 2-cell stage. At stage 17 (A, B), mild abnormalities were observed in the developing neural axis of XSMOC-1 MO-injected embryos (B). By stage 32 (C-E), MO-injected embryos were mildly ventralized (D, E) compared to controls (C). In addition, eyes were absent on the injected side (E); this was more apparent by stage 38 (G). Eye development appeared normal on the non-injected side (F). The XSMOC-1 MO phenotypes shown in (D and E) were typical for this stage and were observed in 90% of the embryos in five independent experiments (n=164). (H-K) Whole mount hybridization in situ analyses of Otx2 (H, I) and Tbx2 (J, K) in stage 32 control (G, J) and XSMOC-1 MO-injected (I, K) embryos. The injected sides are displayed on the right. Arrows indicate the location of the eye fields.

FIG. 6 shows that complete loss of XSMOC-1 function leads to developmental arrest prior to neurulation. (A-F) Embryos injected bilaterally at the two-cell stage with 6ng of 5 base mismatch control (A-C), or antisense (D-F) XSMOC-1MO. Control-MO injected embryos developed normally (the position of the neural tube is indicated in C by an arrow), whereas antisense-MO injected embryos appeared normal up to the end of gastrulation (stage 12), but arrested prior to neurulation. The XSMOC-1 MO phenotypes shown in (F) were typical for this stage and were observed in 95% of the embryos in eight independent experiments (n=326). (G-I) RT-PCR analyses of markers expressed by control and antisense-XSMOC-1 MO-injected embryos at stage 10.5(G), 12(H), and 15(I). Marker expression appeared normal up to stage 12 (G, H), but markers normally expressed after gastrulation were diminished (I).

FIG. 7 depicts whole mount hybridization in situ of control (A, C, E, G, and I) and antisense XSMOC-1 MO-injected (B, D, F, H, and J) embryos showing expression of: XNot (A, B) and XMyf5 (C, D) in stage 11 to 11.5 embryos, XSox2 (E, F) and XNot (G, H) in stage 12.5 embryos, and XSox2 (I, J) in stage 15 embryos. (K, L) Histological sections through I and J showing absence of archenteron (a) and any recognizable dorsal structures in antisense XSMOC-1 MO-injected embryos (modified Von Gieson stain).

FIG. 8 shows that XSMOC-1 inhibits BMP2 activity, but not by direct ligand binding. (A) Embryos were injected bilaterally at the two-cell stage with 360 pg GFP (control), 60 pg BMP2+300 pg GFP, or 60 pg BMP2+300 pg XSMOC-1 mRNAs and incubated until stage 26. In three independent experiments, BMP2 injected embryos were ventralized (82%≦DAI 1, n=84), whereas those co-injected with BMP2 and XSMOC-1 showed partial to complete rescue (70%≧DAI 3AI 3 or greater, n =98). (B, C) RT-PCR analysis of animal cap explants removed from embryos at stage 8 and cultured until sibling embryos reached stage 17. (B) RT-PCR for the ventral marker XVent1 was induced in caps overexpressing BMP2 but not in control injected caps or caps co-expressing BMP2 and XSMOC-1. (C) RT-PCR analysis of animal cap explants removed from embryos injected bilaterally at the two-cell stage with 400 pg GFP (control), 100 pg Activin+300 pg GFP, or 100 pg Activin+300 pg XSMOC-1 mRNAs incubated until stage 17. Expression of the mesodermal marker Brachyury (Bra), induced in caps overexpressing Activin, was not inhibited by co-expression of XSMOC-1. (D) Immunoblot analysis of mouse 3T3 fibroblast cell lysates. 3T3 fibroblasts were transfected with or without XSMOC-1 and exposed to BMP2 for 1 hour. Phosphorylation of Smad1, 5, 8 by BMP2 was blocked in cells transfected with XSMOC-1. (E) RT-PCR analysis of animal cap explants removed from embryos injected bilaterally at the two-cell stage with 450 pg GFP (control), 150 pg caBMPRIB+300 pg GFP, or 150 pg caBMPRIB+300 pg XSMOC-1 mRNAs were incubated until stage 17. The expression of the ventral marker XVent1, induced by overexpression of constitutively active BMP receptor IB (caBMPRIB), was blocked by co-expression with XSMOC-1, but not by noggin.

FIG. 9 shows that XSMOC-1 signals through the MAPK pathway. (A) XSMOC-1 activity was blocked by co-expression of LM-Smad1. RT-PCR analysis of animal caps from embryos injected bilaterally at the two cell stage with 300 pg XSMOC-1+600 pg GFP, 300 pg XSMOC-1+600 pg LM-Smad1, 6 pg noggin+900 pg GFP or 6 pg noggin+600 pg LM-Smad1+300 pg GFP mRNAs. Induction of the neural markers N-CAM, NRP1, and Otx2 by overexpression of XSMOC-1 was blocked by co-expression of LM-Smad1; expression of the epidermal marker keratin was maintained. Neural marker induction (and suppression of keratin) by overexpression of noggin was not affected by co-expression of LM-Smad1. (B) Immunoblot analysis of animal cap extracts from embryos overexpressing XSMOC-1 revealed elevated levels of diphospho-ERK (dp-ERK). Equivalent amounts of protein (10 mg) were loaded per lane. (C) Anterior views of control (left) and XSMOC-1 MO-injected stage 12.5 embryos immunostained for dp-ERK, Note the absence of dp-ERK in XSMOC-1 MO-injected embryo. (D) RT-PCR analysis of animal caps from XSMOC-1-injected embryos incubated in the presence or absence of the MAPK/ERK kinase (MEK) inhibitor U0126 (50 mM) until control embryos reached stage 17. Anterior neuroectodermal (Otx2 and XAG-1) and panneural (NCAM and NRP-1) markers induced by XSMOC-1 were markedly reduced in the presence of U0126.

FIG. 10 is a schematic representation depicting the known and unknown molecular interactions resulting in negative regulation of BMP signaling by XSMOC-1. The diagram shows the BMP Receptor Serine/Threonine Kinase (RS/TK); the Fibroblast Growth Factor (FGF), Epidermal Growth Factor (EGF) and Insulin-like Growth factor (IGF) Receptor Tyrosine Kinase (RTK); Integrin-Linked Kinase (ILK); and Mitogen Activated Protein Kinase (MAPK).

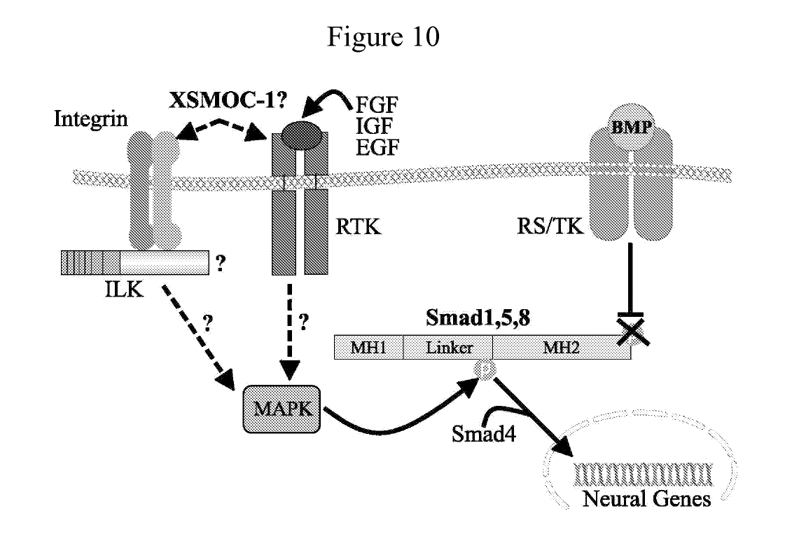

FIG. 11 is an alignment of human (SEQ ID NO:16) and Xenopus (SEQ ID NO:17) SMOC-1 illustrating the conservation of domain structure between human and Xenopus SMOC-1. A consensus sequence (SEQ ID NO:18) is also shown.

### DETAILED DESCRIPTION

Embodiments of the invention relate generally to the field of cell growth, differentiation, and formation of anatomic patterns in a vertebrate subject. Certain aspects of the invention provide methods for modulating skeletal development and growth and differentiation of bone and cartilage for the treatment of disease or tissue damage in a vertebrate subject. Particular aspects of the invention are directed to methods for modulating bone morphogenetic protein (BMP) signaling activity in a cell or tissue of a vertebrate subject that comprise administering to the subject a secreted modular calcium binding protein (SMOC) polypeptide, or a conservatively modified variant, derivative, or analog thereof, or administering a polynucleotide encoding a SMOC polypeptide, or a conservatively modified variant, derivative, or analog thereof, in an amount effective to activate intracellular mitogen activated protein (MAP) kinase activity and to reduce BMP signaling activity in a cell or tissue of the subject.

Certain embodiments of the invention relate to methods for modulating bone morphogenetic protein activity that comprise activating an extracellular signal-regulated mitogen-activated protein kinase with a secreted modular calcium binding protein.

Other embodiments of the invention relate to methods for modulating the growth and differentiation of bone and cartilage in a patient that comprise administering to the patient an effective amount of a secreted modular calcium binding protein; a nucleic acid encoding a secreted modular calcium binding protein; a vector comprising a nucleic acid encoding a secreted modular calcium binding protein; or a host cell expressing a secreted modular calcium binding protein.

Still further embodiments of the invention relate to methods for treating a musculoskeletal disorder comprising administering to a patient suffering from such a disorder a therapeutically effective amount of a secreted modular calcium binding protein; a nucleic acid encoding a secreted modular calcium binding protein; a vector comprising a nucleic acid encoding a secreted modular calcium binding protein; or a host cell expressing a secreted modular calcium binding protein. In preferred aspects of such embodiments of the invention, the musculoskeletal disorder is a joint disorder. In particularly preferred aspects of such embodiments of the invention, the joint disorder is spondylarthropathic disease.

In particular aspects of such embodiments of the invention, the secreted modular calcium binding protein is human secreted modular calcium binding protein-1 or human secreted modular calcium binding protein-2. In preferred aspects of such embodiments of the invention, the human secreted modular calcium binding protein-1 comprises the amino acid sequence of SEQ ID NO:2 or a biologically active fragment or conservatively modified variant thereof. In other preferred aspects of such embodiments of the invention, the human secreted modular calcium binding protein-1 comprises an amino acid sequence that is at least 80%, 81%, 82%, 83%, 84%, 85%, 86%, 87%, 88%, 89%, 90%, 91%, 92%, 93%, 94% 95%, 96%, 97%, 98%, or 99% identical to the amino acid sequence of SEQ ID NO:2, or a biologically active fragment or conservatively modified variant thereof. In particularly preferred embodiments of such aspects of the invention, the human secreted modular calcium binding protein-1 comprises the amino acid sequence of SEQ ID NO:2. In further aspects of such embodiments of the invention, the secreted modular calcium binding protein is Xenopus laevis secreted modular calcium binding protein. In preferred aspects of such embodiments, the Xenopus laevis secreted modular calcium binding protein comprises the amino acid sequence of SEQ ID NO:4 or a biologically active fragment or conservatively modified variant thereof. In other preferred aspects of such embodiments of the invention, the Xenopus laevis secreted modular calcium binding protein comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO:4, or a biologically active fragment or conservatively modified variant thereof. In particularly preferred aspects of such embodiments, the Xenopus laevis secreted modular calcium binding protein comprises the amino acid sequence of SEQ ID NO:4.

In further aspects of such embodiments of the invention, the nucleic acid encoding a secreted modular calcium binding protein encodes human secreted modular calcium binding protein-1 or human secreted modular calcium binding protein-2. In preferred aspects of such embodiments of the invention, the nucleic acid encoding human secreted modular calcium binding protein-1 comprises a nucleotide sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of SEQ ID NO:1. In particularly preferred aspects of such embodiments of the invention, the nucleic acid encoding human secreted modular calcium binding protein-1 comprises the nucleotide sequence of SEQ ID NO:l. In other aspects of such embodiments of the invention, the nucleic acid encoding a secreted modular calcium binding protein encodes Xenopus laevis secreted modular calcium binding protein. In preferred aspects of such embodiments of the invention, the nucleic acid encoding Xenopus laevis secreted modular calcium binding protein comprises a nucleotide sequence that is at least 80%, 81%, 82%, 83%, 84%, 85%, 86%, 87%, 88%, 89%, 90%, 91%, 92%, 93%, 94% 95%, 96%, 97%, 98%, or 99% identical to the nucleotide sequence of SEQ ID NO:3. In particularly preferred aspects of such embodiments of the invention, the nucleic acid encoding Xenopus laevis secreted modular calcium binding protein comprises the nucleotide sequence of SEQ ID NO:3.

The present study investigates a role for the BM-40 family member, Secreted Modular Binding Protein (SMOC), during embryonic development. This study describes the isolation and functional characterization of the Xenopus orthologue of human SMOC-1. SMOC-1 expression could first be detected anteriorly at stage 12.5 at the end of gastrulation and onset of neurulation. In functional assays, XSMOC-1 acted as an antagonist of BMPs. However, unlike other BMP antagonists that act extracellularly by direct ligand binding, XSMOC-1 exerted negative feedback on BMP action through activation of Mitogen Activated Protein (MAP) kinase signaling Inhibition of XSMOC-1 protein expression using an antisense-morpholino oligonucleotide caused complete developmental arrest immediately following gastrulation and prior to formation of the neural plate.

Biochemical studies together with the co-purification of SMOC with BMPs (Chang et al., J Biol Chem 269: 28227-28234, 1994), suggested the possibility of a functional role for SMOC during embryonic development. Xenopus provides a powerful system in which to examine gene function by both gain and loss of function. Isolation of the Xenopus orthologue of human SMOC-1 provided an opportunity to explore its function in Xenopus embryos. In gain-of-function assays, SMOC acted as a BMP antagonist and loss-of-function studies revealed SMOC to be essential for post-gastrulation development.

It is to be understood that this invention is not limited to particular methods, reagents, compounds compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a cell” includes a combination of two or more cells, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“TGF-β superfamily” refers to a family of structurally related growth factors, all of which possess physiologically important growth-regulatory and morphogenetic properties. This family of related growth factors is well known in the art (Kingsley et al., Genes Dev. 8: 133-46, 1994; and Hoodless et al., Curr. Topics Microbiol. Immunol. 228: 235-72, 1998). The TGF-β superfamily includes Bone Morphogenetic Proteins (BMPs), Activins, Inhibins, Mullerian Inhibiting Substance, Glial-Derived Neurotrophic Factor, and a still growing number of Growth and Differentiation Factors (GDFs), such as GDF-5.

“Bone morphogenetic protein” or “BMP” refers to a protein belonging to the BMP family of the TGF-13 superfamily of proteins defined on the basis of DNA and amino acid sequence homology. According to this invention, a protein belongs to the BMP family when it has at least 50% (e.g., at least 70% or even 85%) amino acid sequence similarity or given identity with a known BMP family member within the conserved C-terminal cysteine-rich domain that characterizes the BMP family. Members of the BMP family can have less than 50% DNA or amino acid sequence similarity or identity overall. BMPs act to induce the differentiation of mesenchymal-type cells into chondrocytes and osteoblasts before initiating bone formation, among other functions. Some BMPs act to induce differentiation of cartilage- and bone-forming cells near sites of fractures but also at ectopic locations. Some of the proteins induce the synthesis of alkaline phosphatase and collagen in osteoblasts. Some BMPs act directly on osteoblasts and promote their maturation while at the same time suppressing myogenous differentiation. Other BMPs promote the conversion of mesenchymal cells into chondrocytes and are capable also of inducing the expression of an osteoblast phenotype in non-osteogenic cell types. Most BMPs affect morphogenesis over the entire body, and also affect various repair and pathologic processes in the adult.

“Secreted Modular Calcium Binding Protein (SMOC)” includes the proteins SMOC-1 and SMOC-2. SMOC-1 is a member of the BM-40 family of proteins that are defined by containing a follistatin-like (FS) domain, a pair of thyroglobulin-like (TY) domains, an extracellular calcium-binding (EC) domain, and a novel domain without homology to known proteins. The modular structure of this family is expanded in testicans and tsc36 where additional domains such as the thyroglobulin-like (TY) domain or a domain with partial similarity to van Willebrand factor type C domains have been inserted during evolution. The domain organization of SMOC-1 shows a further variation of this modular setup: SMOC-1 contains one FS, one EC, two TY domains, and a novel domain without known homologs. In all other members of the BM-40 protein family the FS domain is immediately followed by the EC domain, and both domains interact via a small surface (Hohenester et al., EMBO J 16: 3778-3786, 1997). Although the interaction of the FS domain with the EC domain influences calcium binding to the latter, the EC domain is functional and assumes the same structure when expressed separately (Busch, E., et al., J. Biol. Chem. 275: 25508-25515, 2000; Hohenester, E., et al., Nat. Struct. Biol. 3: 67-73, 1996). In SMOC-1 the FS and the EC domain are separated by the two TY domains which are themselves split by the novel domain (Vannahme et al., J Biol Chem 277: 37977-37986, 2002). A similar modular structure involving TY and EC domains is found in the testican family of proteoglycans (Novinec et al., Mol. Biol. Evol. 23, 744-755, 2006).

“Morphogenesis protein” refers to a protein having morphogenesis activity. For instance, such a protein is capable of inducing progenitor cells to proliferate and/or to initiate differentiation pathways that lead to the formation of cartilage, bone, tendon, ligament, neural or other types of tissue, depending on local environmental cues. Thus, morphogenesis proteins useful in this invention can behave differently in different surroundings. A morphogenesis protein of the invention can comprise at least one polypeptide belonging to the SMOC family. Preferred morphogenesis proteins of the invention include hSMOC-1 and hSMOC-2. Particularly preferred is a hSMOC-1 variant comprising the nucleotide sequence of SEQ ID NO: 1 and the amino acid sequence of SEQ ID NO: 2, or a conservatively modified variant, derivative or analog thereof.

“Morphogenesis activity,” “inducing activity” and “tissue inductive activity” alternatively refer to the ability of an agent to stimulate a target cell to undergo one or more cell divisions (proliferation) that can optionally lead to cell differentiation. Such target cells are referred to generically herein as progenitor cells. Cell proliferation is typically characterized by changes in cell cycle regulation and can be detected by a number of means which include measuring DNA synthetic or cellular growth rates, changes in messenger RNA profiles, changes in phosphorylation states or other characteristics associated with the status of signal transduction machinery within the cell. Early stages of cell differentiation are typically characterized by changes in gene expression patterns relative to those of the progenitor cell; such changes can be indicative of a commitment towards a particular cell fate or cell type. Later stages of cell differentiation can be characterized by changes in gene expression patterns, cell physiology, and morphology. Any reproducible change in gene expression, cell physiology, or morphology can be used to assess the initiation, nature and extent of cell differentiation induced by a morphogenic protein.

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm, and ectoderm), as well as to give rise to tissues of multiple germ layers following transplantation and to contribute substantially to most, if not all, tissues following injection into blastocysts.

Stem cells are classified by their developmental potential as: (1) totipotent—able to give rise to all embryonic and extraembryonic cell types; (2) pluripotent—able to give rise to all embryonic cell types; (3) multipotent—able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood); (4) oligopotent—able to give rise to a more restricted subset of cell lineages than multipotent stem cells; and (5) unipotent—able to give rise to a single cell lineage (e.g., spermatogenic stem cells).

Stem cells are also categorized on the basis of the source from which they can be obtained. An adult stem cell is generally a multipotent undifferentiated cell found in tissue comprising multiple differentiated cell types. The adult stem cell can renew itself and, under normal circumstances, differentiate to yield the specialized cell types of the tissue from which it originated, and possibly other tissue types. An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells can be blood-derived (e.g., as are those obtained from umbilical cord blood) or non-blood-derived (e.g., as obtained from the non-blood tissues of the umbilical cord and placenta).

Embryonic tissue is typically defined as tissue originating from the embryo (which in humans refers to the period from fertilization to about six weeks of development. Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac, and allantois), umbilical cord, and placenta (which itself forms from the chorion and the maternal decidua basalis).

Differentiation is the process by which an unspecialized (“uncommitted”) or less specialized cell acquires the features of a specialized cell, such as a nerve cell or a muscle cell, for example. A differentiated or differentiation-induced cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the origin of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

In a broad sense, a progenitor cell is a cell that has the capacity to create progeny cells that are more differentiated than itself and yet retain the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell can be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

A “chondrocyte progenitor cell,” as used herein, refers to a pluripotent, or lineage-uncommitted, progenitor cell that is potentially capable of an unlimited number of mitotic divisions to either renew its line or to produce progeny cells that will differentiate into chondrocytes. This cell is typically referred to as a “stem cell” or “mesenchymal stem cell” in the art. Alternatively, a “chondrocyte progenitor cell” is a lineage-committed progenitor cell produced from the mitotic division of a stem cell that will eventually differentiate into a chondrocyte. The lineage-committed progenitor cell is generally incapable of an unlimited number of mitotic divisions and will eventually differentiate into a chondrocyte. Chondrocyte progenitor cells can come from the synovium or bone marrow, if the subchondral bone plate is penetrated, or other tissues.

“Skeletal tissue” includes cartilage, bone, ligament, or tendon.

Unless defined otherwise, “cartilage,” “bone,” “ligaments,” “tendons,” “synovium,” “periosteum,” “perichondrium”, and related words have their standard meaning

“Cartilage” refers to elastic, translucent connective tissue in mammals, including human and other species. Cartilage is composed predominantly of chondrocytes, type II collagen, small amounts of other collagen types, other noncollagenous proteins, proteoglycans, and water, and is usually surrounded by a perichondrium, made up of fibroblasts, in a matrix of type I and type II collagen as well as other proteoglycans. Although most cartilage becomes bone upon maturation, some cartilage remains in its original form in locations such as the joints, nose, ears, knees, and between intervertebral disks. Cartilage has no blood or nerve supply and chondrocytes are the only type of cell in this tissue.

The function of bone is to provide mechanical support for joints, tendons and ligaments, and to protect vital organs from damage. Bone cells include osteoblasts, the so-called Bone Lining Cells (BLCs), osteocytes, osteoclasts, and other cell types. Osteoblasts are typically viewed as bone forming cells. They are located near to the surface of bone and their functions are to make osteoid and manufacture hormones such as prostaglandins that act on bone itself. Osteoblasts are mononucleate. Active osteoblasts are situated on the surface of osteoid seams and communicate with each other via gap-junctions. Bone Lining Cells (BLCs) share a common lineage with osteogenesis (bone forming) cells. They are flattened, mononucleate cells which line bone. Osteocytes originate from osteoblasts that have migrated into and become trapped and surrounded by bone matrix, which they themselves produce. The spaces that they occupy are known as lacunae. Osteocytes have many processes, which reach out to meet osteoblasts, probably for the purposes of communication. Their functions include formation of bone, matrix maintenance, and calcium homeostasis. Osteocytes possibly act as mechano-sensory receptors regulating the bone's response to mechanical stress.

Tissues connecting bones and muscles are collectively referred to as “connective tissues.” Ligaments are short bands of tough fibrous connective tissue composed mainly of long, stringy collagen molecules. Ligaments generally connect bones to other bones in joints. Tendons are fibrous connective tissues, attached on one end to a muscle and on the other to a bone. The “synovium” or “synovial membrane” is a thin layer of tissue that lines the non-cartilaginous surfaces within the joint space, sealing it from the surrounding tissue. The membrane contains a fibrous outer layer, as well as an inner layer that is responsible for the production of specific components of synovial fluid, which nourishes and lubricates the joint. By “synovial cells” is meant cells derived from the synovium. “Periosteum” refers to the membrane of fibrous connective tissue that closely invests all bones except at the articular surfaces. By “periosteal cells” is meant cells derived exclusively from the periosteum. Periosteal cells can be separated from the periosteum by well-known techniques in the art; subjecting periosteal tissue to trypsinization is but one of many examples for obtaining periosteal cells. The cells, once released from the periosteum or periosteal tissue, can then be grown in cell culture. “Perichondrium” refers to the membrane of connective tissue covering the surface of cartilage except at the articular surfaces. The perichondrium nourishes the avascular cartilage, and it also contains cells including mesenchymal cells, which can differentiate into chondroblasts.

“Cell culture” refers generally to cells taken from a living organism and grown under controlled conditions (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times can be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there can be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, and time between passaging.

A conditioned medium is a medium in which a specific cell or population of cells has been cultured, and then removed. While the cells are cultured in the medium, they secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes survival, growth, proliferation, maintenance, differentiation, and/or maturation of a cell, or stimulates increased activity of a cell.

“Standard growth conditions”, as used herein, refers to culturing of cells (e.g., mammalian cells) at 37° C., in a standard atmosphere comprising 5% CO₂. Relative humidity is maintained at about 100%. While the foregoing the conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like. For example, “standard growth conditions” for yeast (e.g., S. cerevisiae) include 30° C. and generally under regular atmospheric conditions (less than 0.5% CO₂, approximately 20% O₂, approximately 80% N₂) at a relative humidity at about 100%.

“Gene” refers to a unit of inheritable genetic material found in a chromosome, such as in a human chromosome. Each gene is composed of a linear chain of deoxyribonucleotides, which can be referred to by the sequence of nucleotides forming the chain. Thus, “sequence” is used to indicate both the ordered listing of the nucleotides that form the chain, and the chain that has that sequence of nucleotides. The term “sequence” is used in the same way in referring to RNA chains, linear chains made of ribonucleotides. The gene includes regulatory and control sequences, sequences that can be transcribed into an RNA molecule, and can contain sequences with unknown function. Some of the RNA products (products of transcription from DNA) are messenger RNAs (mRNAs), which initially include ribonucleotide sequences (or sequence) that are translated into a polypeptide and ribonucleotide sequences that are not translated. The sequences that are not translated include control sequences, introns, and sequences with unknown function. It can be recognized that small differences in nucleotide sequence for the same gene can exist between different persons, or between normal cells and cancerous cells, without altering the identity of the gene.

“Isolated,” when applied to a nucleic acid or protein, denotes that the nucleic acid or protein is essentially free of other cellular components with which it is associated in the natural state. It is preferably in a homogeneous state although it can either be dry or in an aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19: 5081, 1991; Ohtsuka et al., J. Biol. Chem. 260: 2605-2608, 1985); and Cassol et al., 1992; Rossolini et al., Mol. Cell. Probes 8: 91-98, 1994). For arginine and leucine, modifications at the second base can also be conservative. The term nucleic acid is used interchangeably with gene, cDNA, and mRNA encoded by a gene.

As used herein a “nucleic acid probe” is defined as a nucleic acid capable of binding to a target nucleic acid (e.g., a nucleic acid associated with cancer) of complementary sequence through one or more types of chemical bonds, usually through complementary base pairing, usually through hydrogen bond formation. As used herein, a probe can include natural (i.e., A, G, C, or T) or modified bases (7-deazaguanosine, inosine, and the like). In addition, the bases in a probe can be joined by a linkage other than a phosphodiester bond, so long as it does not interfere with hybridization. Thus, for example, probes can be peptide nucleic acids in which the constituent bases are joined by peptide bonds rather than phosphodiester linkages. It will be understood by one of skill in the art that probes can bind target sequences lacking complete complementarity with the probe sequence depending upon the stringency of the hybridization conditions.

Nucleic acid probes can be DNA or RNA fragments. DNA fragments can be prepared, for example, by digesting plasmid DNA, or by use of PCR, or synthesized by either the phosphoramidite method described by Beaucage and Carruthers, Tetrahedron Lett. 22: 1859-1862, 1981) (Beaucage and Carruthers), or by the triester method according to Matteucci et al., J. Am. Chem. Soc. 103: 3185, 1981) (Matteucci), both incorporated herein by reference. A double stranded fragment can then be obtained, if desired, by annealing the chemically synthesized single strands together under appropriate conditions, or by synthesizing the complementary strand using DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

A “labeled nucleic acid probe” is a nucleic acid probe that is bound, either covalently, through a linker, or through ionic, van der Waals, or hydrogen bonds to a label such that the presence of the probe can be detected by detecting the presence of the label bound to the probe.

The phrase “a nucleic acid sequence encoding” refers to a nucleic acid that contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons that encode a single amino acid) of the native sequence or sequences that can be introduced to conform with codon preference in a specific host cell.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA, DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

Polypeptides can be composed of amino acids joined to each other by peptide bonds or modified peptide bonds, i.e., peptide isosteres, and can contain amino acids other than the 20 gene-encoded amino acids. The polypeptides can be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides can be branched, for example, as a result of ubiquitination, and they can be cyclic, with or without branching. Cyclic, branched, and branched cyclic polypeptides can result from posttranslation natural processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2^{nd} Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182: 626-646, 1990; Rattan et al., Ann N.Y. Acad Sci 663: 48-62, 1992).

Polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Polypeptides can be in the form of the secreted protein, including the mature form, or can be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

Polypeptides are preferably provided in an isolated form, and preferably are substantially purified. A recombinantly produced version of a polypeptide, including the secreted polypeptide, can be substantially purified using techniques described herein or otherwise known in the art, such as, for example, by the one-step method described in Smith and Johnson, Gene 67: 31-40, 1988. Polypeptides of the invention also can be purified from natural, synthetic, or recombinant sources using techniques described herein or otherwise known in the art, such as, for example, antibodies of the invention raised against the polypeptides of the present invention using methods well known in the art.

“Ortholog” refers to an evolutionarily conserved bio-molecule represented in a species other than the organism in which a reference sequence is identified, and contains a nucleic-acid or amino-acid sequence that is homologous to the reference sequence. To determine the degree of similarity between a reference sequence and a sequence in question, two nucleic-acid sequences or two amino-acid sequences are compared. Homology can be defined by testing percentage identity or percentage similarity for statistical significance. Percentage identity reflects the proportion of identical amino-acid residues shared between two sequences compared in an alignment. Percentage similarity correlates with the proportion of amino-acid residues having similar structural properties that is shared between two sequences compared in an alignment. Percentages of similarity and identity can be calculated over a portion of the primary structure and not over the entire gene/protein sequence. For example, amino-acid residues having similar structural properties can be substituted for one another, such as the substitutions of analogous hydrophilic amino-acid residues, and the substitution of analogous hydrophobic amino-acid residues. Percentages of similarity and identity can be calculated over a portion of the primary structure and not over the entire gene/protein sequence. For the present disclosure, an ortholog or an orthologous sequence is defined as a homologous molecule or a sequence that directs the formation of normal joint structures including but not limited to cartilage, ligaments, and tendons and a sequence identity of at least about 20%, 25%, 30%, 35%, 40%, 45%, 50%, 55%, 60%, 65%, 70%, 75%, 80%, 85%, 90%, and 95%. Alternatively, an ortholog is defined as a homologous molecule or sequence that directs the formation of normal joint structures including but not limited to cartilage, ligaments, and tendons and a sequence similarity of at least about 40%, 40%, 45%, 50%, 55%, 60%, 65%, 70%, 75%, 80%, 85%, 90%, and 95%,

It is further contemplated that “ortholog” is a polypeptide or nucleic acid molecule of an organism that is highly related to a reference protein, or nucleic acid sequence, from another organism. An ortholog is functionally related to the reference gene, protein or nucleic acid sequence. In other words, the ortholog and its reference molecule would be expected to fulfill similar, if not equivalent, functional roles in their respective organisms. It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains can be defined by genetic mutations or by structure-function assays. Orthologs can be identified using methods provided herein. The functional role of an ortholog can be assayed using methods well known to the skilled artisan, and described herein. For example, function might be assayed in vivo or in vitro using a biochemical, immunological, or enzymatic assay; transformation rescue, or for example, in a nematode bioassay for the effect of gene inactivation on nematode phenotype. Other model organisms, such as flies, amphibians, fish, and mice afford assays for the effects of gain or loss of gene function as well as the biological distribution gene or protein expression; each of these systems provides specific capabilities. Alternatively, bioassays can be carried out in tissue culture; function can also be assayed by gene inactivation (e.g., by RNAi, siRNA, or gene knockout), or gene over-expression, as well as by other methods.

“Paralogs” are distinct but structurally related proteins made by an organism. Paralogs are believed to arise through gene duplication.

“Variant” can refer to an organism with a particular genotype in singular form, a set of organisms with different genotypes in plural form, and also to alleles of any gene identifiable by methods of the present invention. For example, the term “variants” includes various alleles that can occur at high frequency at a polymorphic locus, and includes organisms containing such allelic variants. The term “variant” includes various “strains” and various “mutants.”

A “wild type protein” or “native protein” comprises a polypeptide having the same amino acid sequence as a protein derived from nature. Thus, a wild type protein can have the amino acid sequence of a naturally occurring rat protein, murine protein, human protein, or protein from any other species. Such wild type SMOC-1 polypeptides and orthologs thereof can be isolated from nature or can be produced by recombinant or synthetic means. The term “wild type protein” specifically encompasses naturally-occurring truncated forms of the protein, naturally-occurring variant forms (e.g., alternatively spliced forms), and naturally-occurring allelic variants of the particular proteins disclosed herein.

“Naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

An intact “antibody” comprises at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) through cellular receptors such as Fc receptors (e.g., FcγRI, FcγRIIa, FcγRIIb, FcγRIII, and FcRr_{π}) and the first component (Clq) of the classical complement system. The term antibody includes antigen-binding portions of an intact antibody that retain capacity to bind the antigen. Examples of antigen binding portions include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341: 544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 242: 423-426, 1988; and Huston et al., Proc. Natl. Acad. Sci. U.S.A. 85: 5879-5883, 1988). Such single chain antibodies are included by reference to the term “antibody” Fragments can be prepared by recombinant techniques or enzymatic or chemical cleavage of intact antibodies.

“Substantially pure” or “isolated” means an object species (e.g., a nucleic acid or polypeptide of the invention) has been identified and separated and/or recovered from a component of its natural environment such that the object species is the predominant species present (e.g., on a molar basis it is more abundant than any other individual species in the composition); a “substantially pure” or “isolated” composition also means where the object species comprises at least about 50 percent (on a molar basis) of all macromolecular species present. A substantially pure or isolated composition can also comprise more than about 80 to 90 percent by weight of all macromolecular species present in the composition. An isolated object species (e.g., a nucleic acid or polypeptide of the invention) can also be purified to essential homogeneity (contaminant species cannot be detected in the composition by conventional detection methods) wherein the composition consists essentially of derivatives of a single macromolecular species. For example, an isolated nucleic acid or polypeptide of any one morphogenic gene product as contemplated herein can be substantially free of other nucleic acids or polypeptides that lack binding to that particular gene product and bind to a different antigen. Further, an isolated nucleic acid or polypeptide that specifically binds to an epitope, isoform or variant of a protein of the invention, can, however, have cross-reactivity to other related antigens, e.g., from other species (e.g., SMOC-1 species homologs). Moreover, an isolated nucleic acid or polypeptide of the invention should be substantially free of other cellular material and/or chemicals.

“Specific binding” refers to preferential binding of a polypeptide to a specified protein relative to other non-specified proteins. The phrase “specifically (or selectively) binds” to an antibody refers to a binding reaction that is determinative of the presence of the protein in a heterogeneous population of proteins and other biologics. Typically, the polypeptide binds with an association constant (Kₐ) of at least about 1×10⁶ M⁻¹ or 10⁷ M⁻¹, or about 10⁸ M⁻¹ to 10⁹ M⁻¹, or about 10¹⁰ M⁻¹ to 10¹¹ M⁻¹ or higher, and binds to the specified protein with an affinity that is at least two-fold, preferable at least ten-fold, and more preferably at least 100-fold greater than its affinity for binding to a non-specific protein (e.g., BSA, casein) other than the specified protein or a closely-related protein..

“Specifically bind(s)” or “bind(s) specifically”, when referring to a peptide, refers to a peptide molecule that has intermediate or high binding affinity, exclusively or predominately, to a target molecule. The phrase “specifically binds to” refers to a binding reaction that is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA, immunoprecipitation, Biacore, and Western blot are used to identify peptides that specifically react with the antigen. Typically a specific or selective reaction will be at least twice background signal or noise and more typically more than 10 times background.

“Substantially identical,” in the context of two nucleic acids or polypeptides refers to two or more sequences or subsequences that have at least about 80%, about 90%, about 95% or higher nucleotide or amino acid residue identity, when compared and aligned for maximum correspondence, as measured using the following sequence comparison method and/or by visual inspection. Such “substantially identical” sequences are typically considered to be homologous. The “substantial identity” can exist over a region of sequence that is at least about 50 residues in length, over a region of at least about 100 residues, over a region of at least about 150 residues, or over the full length of the two sequences to be compared. As described below, any two antibody sequences can only be aligned in one way, by using the numbering scheme in Kabat. Therefore, for antibodies, percent identity has a unique and well-defined meaning

Amino acids from the variable regions of the mature heavy and light chains of immunoglobulins are designated Hx and Lx respectively, where x is a number designating the position of an amino acid according to the scheme of Kabat, Sequences of Proteins of Immunological Interest (National Institutes of Health, Bethesda, Md., 1987 and 1991). Kabat lists many amino acid sequences for antibodies for each subgroup, and lists the most commonly occurring amino acid for each residue position in that subgroup to generate a consensus sequence. Kabat uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention. An alternative structural definition has been proposed by Chothia, et al., J. Mol. Biol. 196: 901-917, 1987; Chothia, et al., Nature 342: 878-883, 1989; and Chothia, et al., J. Mol. Biol. 186: 651-663, 1989, which are herein incorporated by reference for all purposes.

The nucleic acids or polypeptides of the invention may be present in whole cells, in a cell lysate, or in a partially purified or substantially pure form. A nucleic acid or polypeptide is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis, and others well known in the art (See, e.g., Sambrook, Tijssen, and Ausubel discussed herein and incorporated by reference for all purposes). The nucleic acid sequences of the invention and other nucleic acids used to practice this invention, whether RNA, cDNA, genomic DNA, or hybrids thereof, can be isolated from a variety of sources, genetically engineered, amplified, and/or expressed recombinantly. Any recombinant expression system can be used, including, in addition to bacterial, e.g., yeast, insect, or mammalian systems.

Alternatively, these nucleic acids or polypeptide can be chemically synthesized in vitro. Techniques for the manipulation of nucleic acids, such as, e.g., subcloning into expression vectors, labeling probes, sequencing, and hybridization are well described in the scientific and patent literature, see, e.g., Sambrook, Tijssen, and Ausubel. Nucleic acids can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, such as fluid or gel precipitin reactions, immunodiffusion (single or double), immunoelectrophoresis, radioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

The invention provides a recombinant expression system for endoproteolytic processing of a hSMOC-1 protein variant comprising: a) a first nucleotide sequence encoding a hSMOC-1 protein variant having the amino acid sequence as set forth in SEQ ID NO: 2 or conservative substitution thereof; wherein the nucleotide sequence is operatively linked to transcription controlling nucleotide sequences in a host cell.

The nucleic acid compositions of the present invention, while often in a native sequence (except for modified restriction sites and the like), from either cDNA, genomic DNA, or mixtures, can be mutated variants thereof produced in accordance with standard techniques to provide gene sequences with specified characteristics required for particular applications. For coding sequences, these mutations can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Recombinant host cell” (or simply “host cell”) refers to a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny can not, in fact, be identical to the parent cell, but are still included within the scope of the term “host cell” as used herein.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine, O-phosphothreonine, or O-phosphotyrosine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, or methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids may encode any given protein. For instance, the codons GCA, GCC, GCG, and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence with respect to the expression product, but not with respect to actual probe sequences.

As to amino acid sequences, one of skill will recognize that individual substitutions, deletions, or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A point mutation is one aspect of the invention, that, as discussed above, may be conservative (i.e., a lysine to arginine change).

It is known in the art that in many cases one or more amino acids can be deleted from the N-terminus or C-terminus without substantial loss of biological function. See, e.g., Ron, et al., Biol Chem., 268: 2984-2988, 1993. Accordingly, the present invention provides polypeptides having one or more residues deleted from the amino terminus. Similarly, many examples of biologically functional C-terminal deletion mutants are known (see, e.g., Dobeli, et al., 1988). Accordingly, the present invention provides polypeptides having one or more residues deleted from the carboxy terminus. The invention also provides polypeptides having one or more amino acids deleted from both the amino and the carboxyl termini as described below.

Other mutants in addition to N- and C-terminal deletion forms of the protein discussed above are included in the present invention. Thus, the invention further includes variations of the polypeptides which show substantial SMOC-1 polypeptide activity. Such mutants include deletions, insertions, inversions, repeats, and substitutions selected according to general rules known in the art so as to have little effect on activity. One exemplary, biologically active SMOC-1 mutant is a SMOC-1 polypeptide in which the N-terminal Follistatin domain is deleted.

There are two main approaches for studying the tolerance of an amino acid sequence to change, see, Bowie, et al., Science, 247: 1306-1310, 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. These studies have revealed that proteins are surprisingly tolerant of amino acid substitutions.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a discrete structural unit of the polypeptide and are typically 15 to 350 amino acids long. Examples include domains with enzymatic activity, e.g., a kinase domain. Typical domains are made up of sections of lesser organization such as stretches of β-sheet and α-helices. “Tertiary structure” refers to the complete three-dimensional structure of a polypeptide monomer. “Quaternary structure” refers to the three dimensional structure formed by the noncovalent association of independent tertiary units.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are contemplated here.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Biological samples” refers to any tissue or liquid sample obtained from an organism.

“Patient”, “vertebrate subject”, or “mammalian subject” are used herein and refer to mammals such as human patients and non-human primates, as well as experimental animals such as rabbits, rats, and mice, and other animals. Animals include invertebrates and all vertebrates, e.g., mammals and non-mammals, such as sheep, cows, dogs, cats, avian species, chickens, amphibians, reptiles, osteichthes, or chondrichthes.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with a disease, condition or disorder as described herein. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a disease or disorder associated with a disease, condition or disorder as described herein, but does not yet experience or exhibit symptoms, inhibiting the symptoms of a disease or disorder (slowing or arresting its development), providing relief from the symptoms or side-effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Concomitant administration” of a known drug with a compound of the present invention means administration of the drug and the compound at such time that both the known drug and the compound will have a therapeutic effect or diagnostic effect. Such concomitant administration can involve concurrent (i.e., at the same time), prior, or subsequent administration of the drug with respect to the administration of a compound of the present invention. A person of ordinary skill in the art would have no difficulty determining the appropriate timing, sequence, and dosages of administration for particular drugs and compounds of the present invention.

In general, the phrase “well tolerated” refers to the absence of adverse changes in health status that occur as a result of the treatment and would affect treatment decisions.

“Synergistic interaction” refers to an interaction in which the combined effect of two or more agents is greater than the algebraic sum of their individual effects.

“Chronic” administration refers to administration of the agent(s) in a continuous mode as opposed to an acute mode, so as to maintain the initial therapeutic effect (activity) for an extended period of time. “Intermittent” administration is treatment that is not consecutive without interruption, but rather is cyclic in nature.

“Administering”, “introducing,” “delivering,” “placement,” and “implanting” are used interchangeably herein and refer to the placement of cells of the invention into a subject by a method or route which results in at least partial localization of the regenerative cells at a desired site. The cells can be administered by any appropriate route that results in delivery to a desired location in the subject where at least a portion of the cells or components of the cells remain viable. The period of viability of the cells after administration to a subject can be as short as a few hours, e.g., twenty-four hours, to a few days, to as long as several years.

“Musculoskeletal disease” or “spondylarthropathic disease” or “spondyloarthropathy” refers to inflammatory joint diseases associated with the MHC class I molecule HLA-B27 and clinically similar conditions. The term seronegative spondylarthropathy is used by medical practitioners because this set of conditions may mimic rheumatoid diseases such as rheumatoid arthritis, but serological (blood) tests are typically negative for rheumatoid factor (RhF). Subgroups (with increased HLA-B27 frequency) are: ankylosing spondylitis, Caucasians (AS, 92%); ankylosing spondylitis, African-Americans (AS, 50%); reactive arthritis (Reiter's syndrome) (RS, 60-80%); enteropathic arthritis associated with inflammatory bowel disease (IBD, 60%); psoriatic arthritis (60%); isolated acute anterior uveitis (AAU, iritis or iridocyclitis, 50%); and undifferentiated SpA (USpA, 20-25%). Whipple disease and Behcet disease may also be linked to HLA-B27, as may undifferentiated spondyloarthropathy.

“Inhibitors,” “activators,” and “modulators” of the BMP molecules of the invention (genes their associated gene products in cells) are used to refer to inhibitory, activating, or modulating molecules, respectively, identified using in vitro and in vivo assays for binding or signaling, e.g., ligands, agonists, antagonists, and their homologs and mimetics. The term “modulator” includes inhibitors and activators. Inhibitors are agents that, e.g., bind to, partially or totally block stimulation, decrease, prevent, delay activation, inactivate, desensitize, or down regulate the activity of BMP or other genes or the proteins they encode, e.g., antagonists. Activators are agents that, e.g., bind to, stimulate, increase, open, activate, facilitate, enhance activation, sensitize or up regulate the activity of genes or the proteins they encode, e.g., agonists. Modulators include agents that, e.g., alter the interaction of genes or gene products with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring activated ligands, e.g., with altered activity, as well as naturally occurring and synthetic ligands, antagonists, agonists, small chemical molecules, and the like. Such assays for inhibitors and activators include, e.g., applying putative modulator compounds to a cell expressing a receptor and then determining the functional effects on receptor signaling. Samples or assays comprising activated receptors that are treated with a potential activator, inhibitor, or modulator are compared to control samples without the inhibitor, activator, or modulator to examine the extent of inhibition. Control samples (untreated with inhibitors) can be assigned an activity value of 100% Inhibition of activated samples is achieved when the activity value relative to the control is about 80%, optionally 50% or 25-0%. Activation of sample is achieved when the activity value relative to the control is 110%, optionally 150%, optionally 200-500%, or 1000-3000% higher.

“Pharmaceutically acceptable carrier (or medium)”, which can be used interchangeably with “biologically compatible carrier or medium”, refers to reagents, cells, compounds, materials, compositions, and/or dosage forms which are, within the scope of sound medical judgment, suitable for use in contact with the tissues of human beings and animals without excessive toxicity, irritation, allergic response, or other complication commensurate with a reasonable benefit/risk ratio. As described in greater detail herein, pharmaceutically acceptable carriers suitable for use in the present invention include liquids, semi-solid (e.g., gels) and solid materials (e.g., cell scaffolds). As used herein, the term biodegradable describes the ability of a material to be broken down (e.g., degraded, eroded, dissolved) in vivo. The term includes degradation in vivo with or without elimination (e.g., by resorption) from the body. The semi-solid and solid materials can be designed to resist degradation within the body (non-biodegradable) or they can be designed to degrade within the body (biodegradable, bioerodable). A biodegradable material can further be bioresorbable or bioabsorbable, i.e., it can be dissolved and absorbed into bodily fluids (water-soluble implants are one example), or degraded and ultimately eliminated from the body, either by conversion into other materials or breakdown and elimination through natural pathways.

This invention relies on routine techniques in the field of recombinant genetics. Basic texts disclosing the general methods of use in this invention include Sambrook et al., Molecular Cloning, A Laboratory Manual, 3rd ed., 2001; Kriegler, Gene Transfer and Expression: A Laboratory Manual, 1990; and Ausubel et al., eds., Current Protocols in Molecular Biology, 1994; all of which are herein incorporated by reference for all purposes.

### General Techniques

The nucleic acids used to practice this invention, whether RNA, iRNA, antisense nucleic acid, cDNA, genomic DNA, vectors, viruses or hybrids thereof, or other nucleic acid containing preparations can be isolated from a variety of sources, genetically engineered, amplified, expressed/generated recombinantly. Recombinant polypeptides generated from these nucleic acids can be individually isolated or cloned and tested for a desired activity. Any recombinant expression system can be used, including bacterial, mammalian, yeast, insect, or plant cell expression systems.

Alternatively, these nucleic acids can be synthesized in vitro by well-known chemical synthesis techniques, as described in, e.g., Adams, J. Am. Chem. Soc. 105: 661, 1983; Belousov, Nucleic Acids Res. 25: 3440-3444, 1997; Frenkel, Free Radic. Biol. Med. 19: 373-380, 1995; Blommers, Biochemistry 33: 7886-7896, 1994; Narang, Meth. Enzymol. 68: 90, 1979; Brown Meth. Enzymol. 68: 109, 1979; Beaucage, Tetra. Lett. 22: 1859, 1981; U.S. Pat. No. 4,458,066; Summerton J and Dwight Weller Antisense & Nucleic Acid Drug Development 7:187-195, 1997.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which can be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2^{ND} ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y., 1993.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g., fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat. Genet. 15: 333-335, 1997; yeast artificial chromosomes (YAC); bacterial artificial chromosomes (BAC); P1 artificial chromosomes, see, e.g., Woon, Genomics 50: 306-316, 1998; Pl-derived vectors (PACs), see, e.g., Kern, Biotechniques 23: 120-124, 1997; cosmids, recombinant viruses, phages, or plasmids.

The invention provides fusion proteins and nucleic acids encoding them. A BMP antagonist polypeptide, e.g., hSMOC polypeptide, or a conservatively modified variant, derivative, or analog thereof, of the invention can be fused to a heterologous peptide or polypeptide, such as N-terminal identification peptides that impart desired characteristics, such as increased stability or simplified purification. Peptides and polypeptides of the invention can also be synthesized and expressed as fusion proteins with one or more additional domains linked thereto for, e.g., producing a BMP antagonist peptide, to more readily isolate a recombinantly synthesized peptide, to identify and isolate a BMP antagonist -expressing cell line, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, the domain utilized in the FLAGS extension/affinity purification system (Immunex Corp., Seattle, Wash.), and cleavable linker sequences such as Factor Xa or enterokinase recognition sites (Invitrogen, San Diego, Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site. (See e.g., Williams, Biochemistry 34: 1787-1797, 1995; Dobeli, Protein Expr. Purif 12: 404-414, 1998). The histidine residues facilitate detection and purification, while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell. Biol. 12: 441-53, 1993.

### Peptides and Polypeptides

The invention provides isolated or recombinant polypeptides comprising an amino acid sequence having at least 95%, 96%, 97%, 98%, 99%, or more sequence identity to a sequence of SEQ ID NO: 2, over a region of at least about 100, 150, 200, 250, 300, 350, 400, 450, 500, 550, 600, 650, 700, 750, 800, 850, 900, 950, 1000, 1100, or more residues, or, the full length of the polypeptide, or, a polypeptide encoded by a nucleic acid of the invention. In one aspect, the polypeptide comprises SEQ ID NO: 2. The invention provides methods for inhibiting the activity of morphogenic polypeptides, e.g., a BMP polypeptide. The invention also provides methods for screening for compositions that inhibit the activity of, or bind to (e.g., bind to the active site), of morphogenic polypeptides, e.g., a BMP polypeptide.

In one aspect, the invention provides morphogenic polypeptides (and the nucleic acids encoding them), e.g., hSMOC polypeptide, or a conservatively modified variant, derivative, or analog thereof, where one, some or all of the morphogenic polypeptides are replaced with substituted amino acids. In one aspect, the invention provides methods to disrupt the interaction of morphogenic polypeptides with other proteins, e.g., hSMOC polypeptides.

The peptides and polypeptides of the invention can be expressed recombinantly in vivo after administration of nucleic acids, as described above, or, they can be administered directly, e.g., as a pharmaceutical composition. They can be expressed in vitro or in vivo to screen for modulators of a morphogenic activity and for agents that can ameliorate a musculoskeletal disorder or spondylarthropathic disease. Polypeptides e.g., hSMOC polypeptide, or a conservatively modified variant, derivative, or analog thereof) of the invention can also be used to as a BMP antagonist to treat musculoskeletal disorder in a subject.

Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 433 Peptide Synthesizer in accordance with the instructions provided by the manufacturer.

The peptides and polypeptides of the invention, as defined above, include all “mimetic” and “peptidomimetic” forms. The terms “mimetic” and “peptidomimetic” refer to a synthetic chemical compound that has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, a mimetic composition is within the scope of the invention if, when administered to or expressed in a cell, it has a morphogenic-signaling activity e.g., activity of an hSMOC polypeptide, or a conservatively modified variant, derivative, or analog thereof. A mimetic composition can also be within the scope of the invention if it can inhibit an activity of a morphogenic polypeptide, e.g., be a dominant negative mutant or, bind to an antibody of the invention.

Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds, or other coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC) Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola, Chemistry and Biochemistry of Amino Acids, Peptides and Proteins 7: 267-357, 1983).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′-N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl residues can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanido)-acetic acid, or (guanido)alkyl-acetic acid, where alkyl is defined above. Nitrile derivatives (e.g., containing the CN-moiety in place of COOH) can be substituted for guanido or glutamine. Asparaginyl and glutaminyl residues can be deaminated to the corresponding aspartyl or glutamyl residues.

Arginine residue mimetics can be generated by reacting arginyl residues with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be oxidized to form, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a polypeptide of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form

The invention also provides polypeptides that are “substantially identical” to an exemplary polypeptide of the invention. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine. One or more amino acids can be deleted, for example, from a morphogenic polypeptide of the invention, resulting in modification of the structure of the polypeptide, without significantly altering its biological activity. For example, amino- or carboxyl-terminal, or internal, amino acids that are not required for a morphogenic-signaling activity could be reduced or eliminated.

The skilled artisan will recognize that individual synthetic residues and polypeptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 9: 205-223, 1998; Hruby, Curr. Opin. Chem. Biol. 1: 114-119, 1997; Ostergaard, Mol. Divers. 3: 17-27, 1997; Ostresh, Methods Enzymol. 267: 220-234, 1996. Modified peptides of the invention can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing chondrocyte cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.) and the inclusion of cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (See e.g., Williams, Biochemistry 34: 1787-1797, 1995; Dobeli, Protein Expr. Purif. 12: 404-14, 1998). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see e.g., Kroll, DNA Cell. Biol., 12: 441-53, 1993.

The terms “polypeptide” and “protein” as used herein, refer to amino acids joined to each other by peptide bonds or modified peptide bonds, i.e., peptide isosteres, and can contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidomimetic” forms, as described in further detail, below.

As used herein, the term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. As used herein, an isolated material or composition can also be a “purified” composition, i.e., it does not require absolute purity; rather, it is intended as a relative definition. Individual nucleic acids obtained from a library can be purified conventionally to apparent electrophoretic homogeneity. In alternative aspects, the invention provides nucleic acids that have been purified from genomic DNA or from other sequences in a library or other environment by at least one, two, three, four, five, or more orders of magnitude.

### Fusion Proteins

Antibodies or SMOC polypeptides or derivatives thereof binding to morphogenic gene products (e.g., a morphogenic protein, BMP polypeptide, hSMOC polypeptide, or MAP kinase polypeptide) can be used to generate fusion proteins. For example, the nucleic acids or polypeptides of the present invention, when fused to a second protein, can be used as an antigenic tag. Antibodies raised against a morphogenic gene product (e.g., a morphogenic protein) can be used to detect the second protein indirectly by binding to the polypeptide.

Examples of domains that can be fused to polypeptides include not only heterologous signal sequences, but also other heterologous functional regions. The fusion does not necessarily need to be direct, but can occur through linker sequences.

Moreover, fusion proteins can also be engineered to improve characteristics of the polypeptide. For instance, a region of additional amino acids, particularly charged amino acids, can be added to the N-terminus of the polypeptide to improve stability and persistence during purification from the host cell or subsequent handling and storage. Other fusions might be constructed to direct the polypeptide to particular subcellular compartments. Also, peptide moieties can be added to the polypeptide to facilitate purification. Such regions can be removed prior to final preparation of the polypeptide. The addition of peptide moieties to facilitate handling of polypeptides is a familiar and routine technique in the art.

Moreover, antibody compositions or SMOC polypeptides or derivatives thereof binding to morphogenic proteins, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. EP-A 394,827; Traunecker et al., Nature, 331: 84-86, 1988. Fusion proteins having disulfide-linked dimeric structures (due to the IgG) can also be more efficient in binding and neutralizing other molecules than the monomeric secreted protein or protein fragment alone. Fountoulakis et al., J. Biochem. 270: 3958-3964, 1995.

Similarly, EP-A-0 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion can hinder therapy and diagnosis if the fusion protein is used as an antigen for immunizations. In drug discovery, for example, human proteins, such as hIL-5, have been fused with Fc portions for the purpose of high throughput screening assays to identify antagonists of hIL-5 Bennett et al., J. Molecular Recognition 8: 52-58, 1995; Johanson et al., J. Biol. Chem., 270: 9459-9471, 1995.

Moreover, the polypeptides can be fused to marker sequences, such as a peptide that facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein (Wilson et al., Cell 37: 767, 1984).

Additional fusion proteins of the invention can be generated through the techniques of gene-shuffling, motif-shuffling, exon-shuffling, or codon-shuffling (collectively referred to as “DNA shuffling”). DNA shuffling can be employed to modulate the activities of polypeptides of the present invention thereby effectively generating agonists and antagonists of the polypeptides. See, for example, U.S. Pat. Nos. 5,605,793; 5,811,238; 5,834,252; 5,837,458; Patten, et al., Curr. Opinion Biotechnol., 8: 724-733, 1997; Harayama, Trends Biotechnol., 16: 76-82, 1998; Hansson, et al., J. Mol. Biol., 287: 265-276, 1999; Lorenzo, et al., Biotechniques, 24: 308-313, 1998. (Each of these documents is hereby incorporated by reference). In one embodiment, one or more components, motifs, sections, parts, domains, fragments, and the like, of coding polynucleotides of the invention, or the polypeptides encoded thereby can be recombined with one or more components, motifs, sections, parts, domains, fragments, and the like of one or more heterologous molecules.

Thus, any of these above fusions can be engineered using the polynucleotides or the polypeptides of the present invention.

### Transcriptional Control Elements

The nucleic acids of the invention can be operatively linked to a promoter. A promoter can be one motif or an array of nucleic acid control sequences, that direct transcription of a nucleic acid. A promoter can include necessary nucleic acid sequences near the start site of transcription, such as, in the case of a RNA polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

### Expression Vectors and Cloning Vehicles

The invention provides expression vectors and cloning vehicles comprising nucleic acids of the invention, e.g., sequences encoding the proteins of the invention. Expression vectors and cloning vehicles of the invention can comprise viral particles, baculoviruses, phage, plasmids, phagemids, cosmids, fosmids, bacterial artificial chromosomes, viral DNAs (e.g., vaccinia, adenovirus, fowl pox virus, pseudorabies and derivatives of SV40), P1-based artificial chromosomes, yeast plasmids, yeast artificial chromosomes, and any other vectors specific for specific hosts of interest (such as Bacillus, Aspergillus and yeast). Vectors of the invention can include chromosomal, non-chromosomal, and synthetic DNA sequences, including transposons. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The vector is then used to transform an appropriate host cell. Suitable recombinant expression systems include, but are not limited to, bacterial, mammalian, baculovirus/insect, vaccinia, Semliki Forest virus (SFV), Alphavirus (such as Sindbis or Venezuelan Equine Encephalitis (VEE)), mammalian, yeast, and Xenopus expression systems well known in the art. Particularly preferred expression systems are mammalian cell lines, vaccinia, Sindbis, eucaryotic layered vector initiation systems (e.g., U.S. Pat. No. 6,015,686, U.S. Pat. No. 5, 814,482, U.S. Pat. No. 6,015,694, U.S. Pat. No. 5,789,245, EP 1029068A2, WO 9918226A2/A3, EP 00907746A2, WO 9738087A2, all herein incorporated by reference in their entireties for all purposes), insect, and yeast systems. Other expression systems include autologous or allogeneic human cells. Other expression systems include chondrocyte progenitor cells.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature 328: 731, 1987; Schneider, Protein Expr. Purif. 6435: 10, 1995; Sambrook or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors, or viruses that are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, or into skeletal, bone, or cartilage tissue, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors, which exploit advantageous merits of each of the parent vector properties, can also be employed. (See e.g., Feng, Nature Biotechnology 15: 866-870, 1997). Such viral genomes can be modified by recombinant DNA techniques to include the nucleic acids of the invention, and can be further engineered to be replication deficient, conditionally replicating, or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral, and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher, J. Virol. 66: 2731-2739, 1992; Johann, J. Virol. 66: 1635-1640, 1992). Adeno-associated virus (AAV)-based vectors can be used to infect cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther. 3: 957-964, 1996. See also the Cellular Transfection and Gene Therapy section below.

“Expression cassette” as used herein refers to a nucleotide sequence capable of effecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. For switch sequences, operably linked indicates that the sequences are capable of effecting switch recombination.

“Vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Like retroviruses, transposons and transposon vectors can also be used to integrate sequences that an act as insertional mutagens. Also like retroviruses, transposons integrate by enzymatically catalyzed non-homologous recombination in which transposase enzymes catalyze the genomic integration and transposition of transposon DNA (Cui et al., J Mol Biol. 318: 1221-35, 2002; Izsvak et al., J Biol Chem. 277: 34581-8, Epub 2002 Jun. 24; see also Devine and Boeke, Nucl. Acids Res. 22: 3765-2772, 1994). By “transposon” or “transposable element” is meant a linear strand of DNA capable of integrating into a second strand of DNA which may be linear (e.g., genomic DNA or linearized plasmid) or may be a circularized plasmid.

### Host Cells and Transformed Cells

The invention also provides a transformed cell comprising a nucleic acid sequence of the invention, e.g., a sequence encoding a morphogenic polypeptide of the invention, e.g., hSMOC polypeptide, or a conservatively modified variant, derivative, or analog thereof, or a vector of the invention. The host cell can be any of the host cells familiar to those skilled in the art, including prokaryotic cells, such as bacterial cells, or eukaryotic cells, such as fungal cells including yeast cells, mammalian cells, insect cells, or plant cells. Exemplary bacterial cells include E. coli, Bacillus subtilis, Salmonella typhimurium and various species within the genera Pseudomonas, Streptomyces, and Staphylococcus. Exemplary insect cells include Drosophila S2 and Spodoptera Sf9 cells. Exemplary animal cells include CHO, COS, Bowes melanoma, or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants, or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof

Cells can be harvested by centrifugation, disrupted by physical or chemical means, and the resulting crude extract retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, or other types of adsorption chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa, and BHK cell lines.

The constructs in host cells can be used in a conventional manner to produce the gene product encoded by the recombinant sequence. Depending upon the host employed in a recombinant production procedure, the polypeptides produced by host cells containing the vector can be glycosylated or can be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a characteristic allowing for selection of transformed host cells, such as dihydrofolate reductase or neomycin resistance for eukaryotic cell culture, or such as tetracycline or ampicillin resistance in E. coli.

### Amplification of Nucleic Acids

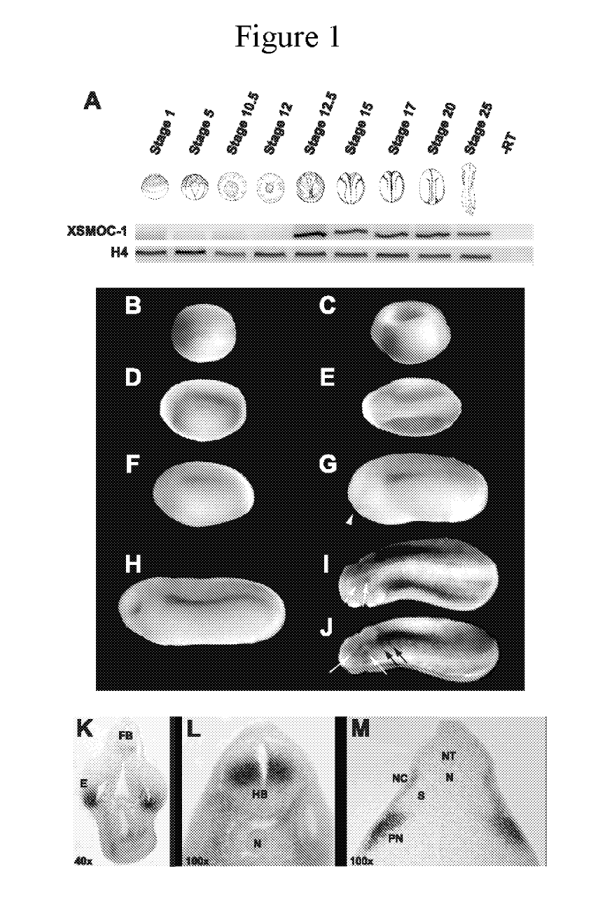

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the exemplary sequences in FIG. 1, or subsequences thereof.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4: 560, 1989; Landegren, Science 241: 1077, 1988; Barringer, Gene 89: 117, 1990); transcription amplification (see, e.g., Kwoh, Proc. Natl. Acad. Sci. USA 86: 1173, 1989); and self-sustained sequence replication (see, e.g., Guatelli, Proc. Natl. Acad. Sci. USA 87: 1874, 1990); Q Beta replicase amplification (see, e.g., Smith, J. Clin. Microbiol. 35: 1477-1491, 1997), automated Q-beta replicase amplification assay (see, e.g., Burg, Mol. Cell. Probes 10: 257-271, 1996) and other RNA polymerase mediated techniques (e.g., NASBA, Cangene, Mississauga, Ontario); see also Berger, Methods Enzymol. 152: 307-316, 1987; Sambrook; Ausubel; U.S. Pat. Nos. 4,683,195 and 4,683,202; Sooknanan, Biotechnology 13: 563-564, 1995.

### Hybridization of Nucleic Acids

The invention provides isolated or recombinant nucleic acids that hybridize under stringent conditions to an exemplary sequence of the invention, e.g., SEQ ID NO: 1, or the complement thereof, or a nucleic acid that encodes a polypeptide of the invention. In alternative aspects, the stringent conditions are highly stringent conditions, medium stringent conditions, or low stringent conditions, as known in the art and as described herein. These methods can be used to isolate nucleic acids of the invention.

In alternative aspects, nucleic acids of the invention as defined by their ability to hybridize under stringent conditions, can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, RNAi, shRNA, antisense oligonucleotides, or sequences encoding antibody binding peptides (epitopes), motifs, active sites and the like.

“Selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention by its ability to hybridize under stringent conditions to a nucleic acid otherwise determined to be within the scope of the invention (such as the exemplary sequences described herein).

“Stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in e.g., Sambrook, ed., Molecular Cloning: A Laboratory Manual (2^{nd} Ed.), Vols. 1-3, Cold Spring Harbor Laboratory, 1989; Current Protocols in Molecular Biology, Ausubel, ed. John Wiley & Sons, Inc., New York, 1997; Laboratory Techniques In Biochemistry And Molecular Biology: Hybridization With Nucleic Acid Probes, Part I. Theory and Nucleic Acid Preparation, Tijssen, ed. Elsevier, N.Y., 1993.

Generally, stringent conditions are selected to be about 5-10° C. lower than the thermal melting point I for the specific sequence at a defined ionic strength and pH. The Tₘ is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide, as described in Sambrook (cited below). For high stringency hybridization, a positive signal is at least two times background, preferably 10 times background hybridization. Exemplary high stringency or stringent hybridization conditions include: 50% formamide, 5×SSC, and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions that determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or equivalent conditions. See Sambrook, Tijssen, and Ausubel for a description of SSC buffer and equivalent conditions.

### Oligonucleotide Probes and Methods for Using Them

The invention also provides nucleic acid probes for identifying nucleic acids encoding a polypeptide that is a BMP modulator, antagonist, or agonist, of a morphogenic-signaling activity. In one aspect, the probe comprises at least 10 consecutive bases of a nucleic acid of the invention, such as, for example, the nucleic acid set forth in SEQ ID NO:1 or its complement. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a nucleic acid of the invention such as, for example, the nucleic acid set forth in SEQ ID NO:1 or its complement. The probes identify a nucleic acid by binding and/or hybridization. The probes can be used in arrays of the invention; see discussion below. The probes of the invention can also be used to isolate other nucleic acids or polypeptides.

### Determining the Degree of Sequence Identity

The invention provides nucleic acids having at least 90%, 91%, 92%, 93%, 94%, 95%, 96%, 97%, 98%, 99% or more sequence identity to the sequences of the present invention as shown in FIG. 1. The invention provides polypeptides having at least 90%, 91%, 92%, 93%, 94%, 95%, 96%, 97%, 98%, 99% or more sequence identity sequences of the present invention as shown in FIG. 1. The sequence identities can be determined by analysis with a sequence comparison algorithm or by a visual inspection. Protein and/or nucleic acid sequence identities and similarities can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2 or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 residues in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biotechnology Information), GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., (1999 Suppl.), Current Protocols in Molecular Biology, Greene Publishing Associates and Wiley Interscience, N.Y., 1987).

A preferred example of an algorithm that is suitable for determining percent sequence identity and sequence similarity is the FASTA algorithm, which is described in Pearson & Lipman, Proc. Natl. Acad. Sci. U.S.A. 85: 2444, 1988. See also Pearson, Methods Enzymol. 266: 227-258, 1996. Preferred parameters used in a FASTA alignment of DNA sequences to calculate percent identity are optimized, BL50 Matrix 15: −5, k-tuple=2; joining penalty=40, optimization=28; gap penalty −12; gap length penalty=−2; and width=16.

Another preferred example of algorithm that is suitable for determining percent sequence identity and sequence similarity are the BLAST and BLAST 2.0 algorithms, which are described in Altschul et al., Nuc. Acids Res. 25: 3389-3402, 1977; and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always<0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, an expectation (E) of 10, and the BLOSUM62 scoring matrix (see Henikoff & Henikoff, Proc. Natl. Acad. Sci. U.S.A. 89: 10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90: 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35: 351-360, 1987. The method used is similar to the method described by Higgins & Sharp, CABIOS 5: 151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0. (Devereaux et al., Nuc. Acids Res. 12: 387-395, 1984).

Another preferred example of an algorithm that is suitable for multiple DNA and amino acid sequence alignments is the CLUSTALW program (Thompson et al., Nucl. Acids. Res. 22: 4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix. (Henikoff and Henikoff, Proc. Natl. Acad. Sci. U.S.A. 89: 10915-10919, 1992).

“Sequence identity” refers to a measure of similarity between amino acid or nucleotide sequences, and can be measured using methods known in the art, such as those described below:

“Identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

“Identity” in the context of two or more nucleic acids or polypeptide sequences, refers to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (e.g., SEQ ID NO: 1 or 2) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. “Homology” refers specifically to whether two sequences share common ancestry (see Doolittle, R. F. (1987) Of URFs and ORFs, University Science Books, Mill Valley), generally based on one or more statistical tests for the significance of the sequence similarity under evaluation.

A “comparison window”, as used herein, includes reference to a segment of any one of the numbers of contiguous residues. For example, in alternative aspects of the invention, contiguous residues ranging anywhere from 20 to the full length of an exemplary polypeptide or nucleic acid sequence of the invention are compared to a reference sequence of the same number of contiguous positions after the two sequences are aligned optimally. If the reference sequence has the requisite sequence identity to an exemplary polypeptide or nucleic acid sequence of the invention, e.g., at least 90%, 91%, 92%, 93%, 94%, 95%, 96%, 97%, 98%, 99% or more sequence identity to the sequences of the invention sequence (e.g., SEQ ID NO: 1 or 2), that sequence is within the scope of the invention.

Motifs that can be detected using the above programs include sequences encoding leucine zippers, helix-turn-helix motifs, glycosylation sites, ubiquitination sites, alpha helices, beta sheets, signal sequences encoding signal peptides, which direct the secretion of the encoded proteins, sequences implicated in transcription regulation, such as homeoboxes, acidic stretches, enzymatic active sites, substrate binding sites, and enzymatic cleavage sites.

### Inhibiting Expression of Polypeptides and Transcripts

The invention further provides for nucleic acids complementary to (e.g., antisense sequences to) the nucleic acid sequences of the invention, such as, for example, the nucleic acid set forth in SEQ ID NO:1 or its complement. Antisense sequences are capable of inhibiting the transport, splicing or transcription of protein-encoding genes, e.g., the BMP antagonist nucleic acids encoding the polypeptides of the invention. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides that are able to either bind gene or message, in either case preventing or inhibiting the production or function of the protein. The association can be through sequence specific hybridization. Another useful class of inhibitors includes oligonucleotides that cause inactivation or cleavage of protein message. The oligonucleotide can have enzyme activity that causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology, and RNAi technology to control gene expression, or of gene therapy methods for expression of an exogenous gene in this manner are well known in the art. Each of these methods utilizes a system, such as a vector, encoding either an antisense or ribozyme transcript of an hSMOC polypeptide, or a conservatively modified variant, derivative, or analog thereof, of the invention. The term “RNAi” stands for RNA interference. This term is understood in the art to encompass technology using RNA molecules that can silence genes. (See, for example, McManus, et al., Nature Reviews Genetics 3: 737, 2002). In this application, the term “RNAi” encompasses molecules such as short interfering RNA (siRNA), microRNAs (mRNA), or small temporal RNA (stRNA). Generally speaking, RNA interference results from the interaction of double-stranded RNA with genes.

### Antisense Oligonucleotides

The invention provides antisense oligonucleotides synthesized by various methods (including, but not limited to, phosphorothioate, morpholino, and peptide nucleic acid chemistries) capable of binding the message encoding the morphogenic polypeptide, which can inhibit polypeptide synthesis by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units, can be used. Antisense oligonucleotides having phosphorothioate linkages can also be used, as described in WO 97/03211; WO 96/39154; Mata, Toxicol Appl Pharmacol 144: 189-197, 1997; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

The invention provides a method of inhibiting expression of a gene encoding a morphogenic protein comprising the steps of (i) providing a biological system in which expression of a gene encoding a morphogenic protein is to be inhibited; and (ii) contacting the system with an antisense molecule that hybridizes to a transcript encoding the morphogenic protein. In other embodiments, morphogenic proteins are inhibited. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression can be inducible and/or tissue or cell type-specific. The antisense molecule can be an oligonucleotide or a longer nucleic acid molecule. The invention provides such antisense molecules.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention. (See, e.g., Gold, J. Biol. Chem. 270: 13581-13584, 1995).

siRNA

RNA interference (RNAi) is a mechanism of post-transcriptional gene silencing mediated by double-stranded RNA (dsRNA), which is distinct from antisense and ribozyme-based approaches (see Jain, Pharmacogenomics 5: 239-42, 2004 for a review of RNAi and siRNA). RNA interference is useful in a method for treating a musculoskeletal disorder or spondylarthropathic disease in a mammal by administering to the mammal a nucleic acid molecule (e.g., dsRNA) that hybridizes under stringent conditions to a morphogenic sequence or MAP kinase sequence as described herein, and attenuates expression of said target gene. dsRNA molecules are believed to direct sequence-specific degradation of mRNA in cells of various types after first undergoing processing by an RNase III-like enzyme called DICER (Bernstein et al., Nature 409: 363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

In general, a short, interfering RNA (siRNA) comprises an RNA duplex that is preferably approximately 19 base pairs long and optionally further comprises one or two single-stranded overhangs or loops. A siRNA can comprise two RNA strands hybridized together, or can alternatively comprise a single RNA strand that includes a self-hybridizing portion. siRNAs can include one or more free strand ends, which can include phosphate and/or hydroxyl groups. siRNAs typically include a portion that hybridizes under stringent conditions with a target transcript. One strand of the siRNA (or, the self-hybridizing portion of the siRNA) is typically precisely complementary with a region of the target transcript, meaning that the siRNA hybridizes to the target transcript without a single mismatch. In certain embodiments of the invention in which perfect complementarity is not achieved, it is generally preferred that any mismatches be located at or near the siRNA termini.

siRNAs have been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, Nat. Biotechnol. 20: 446-448, 2002; See also Yu et al., Proc. Natl. Acad. Sci., 99: 6047-6052, 2002; Sui et al., Proc. Natl. Acad. Sci USA., 99: 5515-5520, 2002; Paddison et al., Genes and Dev. 16: 948-958, 2002; Brummelkamp et al., Science 296: 550-553, 2002; Miyagashi and Taira, Nat. Biotech. 20: 497-500, 2002; Paul et al., Nat. Biotech. 20: 505-508, 2002. As described in these and other references, the siRNA can consist of two individual nucleic acid strands or of a single strand with a self-complementary region capable of forming a hairpin (stem-loop) structure. A number of variations in structure, length, number of mismatches, size of loop, identity of nucleotides in overhangs, and the like, are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it can also be preferable to select sequences complementary to regions within the 3′ portion of the target transcript. Generally it is preferred to select sequences that contain approximately equimolar ratios of the different nucleotides and to avoid stretches in which a single residue is repeated multiple times.

siRNAs can thus comprise RNA molecules having a double-stranded region approximately 19 nucleotides in length with 1-2 nucleotide 3′ overhangs on each strand, resulting in a total length of between approximately 21 and 23 nucleotides. As used herein, siRNAs also include various RNA structures that can be processed in vivo to generate such molecules. Such structures include RNA strands containing two complementary elements that hybridize to one another to form a stem, a loop, and optionally an overhang, preferably a 3′ overhang. Preferably, the stem is approximately 19 by long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and can be up to approximately 29 nucleotides in length. Loops of 4 nucleotides or greater are less likely subject to steric constraints than are shorter loops and therefore can be preferred. The overhang can include a 5′ phosphate and a 3′ hydroxyl. The overhang can but need not comprise a plurality of U residues, e.g., between 1 and 5 U residues. Classical siRNAs, as described above, trigger degradation of mRNAs to which they are targeted, thereby also reducing the rate of protein synthesis. In addition to siRNAs that act via the classical pathway, certain siRNAs that bind to the 3′ UTR of a template transcript can inhibit expression of a protein encoded by the template transcript by a mechanism related to, but distinct from, classic RNA interference, e.g., by reducing translation of the transcript rather than decreasing its stability. Such RNAs are referred to as microRNAs (miRNAs) and are typically between approximately 20 and 26 nucleotides in length, e.g., 22 nt in length. It is believed that they are derived from larger precursors known as small temporal RNAs (stRNAs) or mRNA precursors, which are typically approximately 70 nt long with an approximately 4-15 nt loop (See Grishok et al., Cell 106: 23-24, 2001; Hutvagner et al., Science 293: 834-838, 2001; Ketting, et al., Genes Dev., 15: 2654-2659, 2001). Endogenous RNAs of this type have been identified in a number of organisms including mammals, suggesting that this mechanism of post-transcriptional gene silencing can be widespread (Lagos-Quintana et al., Science 294: 853-858, 2001; Pasquinelli, Trends in Genetics 18: 171-173, 2002, and references in the foregoing two articles). MicroRNAs have been shown to block translation of target transcripts containing target sites in mammalian cells (Zeng et al., Molecular Cell 9: 1-20, 2002).

siRNAs such as naturally occurring or artificial (i.e., designed by humans) mRNAs that bind within the 3′ UTR (or elsewhere in a target transcript) and inhibit translation can tolerate a larger number of mismatches in the siRNA/template duplex, and particularly can tolerate mismatches within the central region of the duplex. In fact, there is evidence that some mismatches can be desirable or required as naturally occurring stRNAs frequently exhibit such mismatches, as do miRNAs that have been shown to inhibit translation in vitro. For example, when hybridized with the target transcript such siRNAs frequently include two stretches of perfect complementarity separated by a region of mismatch. A variety of structures is possible. For example, the miRNA can include multiple areas of nonidentity (mismatch). The areas of nonidentity (mismatch) need not be symmetrical in the sense that both the target and the miRNA include nonpaired nucleotides. Typically the stretches of perfect complementarity are at least 5 nucleotides in length, e.g., 6, 7, or more nucleotides in length, while the regions of mismatch can be, for example, 1, 2, 3, or 4 nucleotides in length.

Hairpin structures designed to mimic siRNAs and mRNA precursors are processed intracellularly into molecules capable of reducing or inhibiting expression of target transcripts (McManus et al., RNA 8: 842-850, 2002). These hairpin structures, which are based on classical siRNAs consisting of two RNA strands forming a 19 by duplex structure, are classified as class I or class II hairpins. Class I hairpins incorporate a loop at the 5′ or 3′ end of the antisense siRNA strand (i.e., the strand complementary to the target transcript whose inhibition is desired) but are otherwise identical to classical siRNAs. Class II hairpins resemble mRNA precursors in that they include a 19 nt duplex region and a loop at either the 3′ or 5′ end of the antisense strand of the duplex in addition to one or more nucleotide mismatches in the stem. These molecules are processed intracellularly into small RNA duplex structures capable of mediating silencing. They appear to exert their effects through degradation of the target mRNA rather than through translational repression as is thought to be the case for naturally occurring mRNAs and stRNAs.

Thus it is evident that a diverse set of RNA molecules containing duplex structures is able to mediate silencing through various mechanisms. For the purposes of the present invention, any such RNA, one portion of which binds to a target transcript and reduces its expression, whether by triggering degradation, by inhibiting translation, or by other means, is considered to be an siRNA, and any structure that generates such an siRNA (i.e., serves as a precursor to the RNA) is useful in the practice of the present invention.

In the context of the present invention, siRNAs are useful both for therapeutic purposes, e.g., to modulate the expression of a morphogenic molecule or protein, or hSMOC polypeptide, or conservatively modified variant, derivative, or analog thereof, in a subject at risk of or suffering from musculoskeletal disorder, or spondylarthropathic disease. In another aspect, the therapeutic treatment of a musculoskeletal target with an antibody, antisense vector, or double stranded RNA vector is also contemplated.

The invention therefore provides a method of inhibiting expression of a gene encoding a morphogenic protein comprising the step of (i) providing a biological system in which expression of a gene encoding morphogenic protein is to be inhibited; and (ii) contacting the system with an siRNA targeted to a transcript encoding the morphogenic protein. In other embodiments, morphogenic proteins, e.g., bone morphogenic proteins, are inhibited. According to certain embodiments of the invention the biological system comprises a cell, and the contacting step comprises expressing the siRNA in the cell. According to certain embodiments of the invention the biological system comprises a subject, e.g., a mammalian subject such as a mouse or human, and the contacting step comprises administering the siRNA to the subject or comprises expressing the siRNA in the subject. According to certain embodiments of the invention the siRNA is expressed inducibly and/or in a cell-type or tissue specific manner.

By “biological system” is meant any vessel, well, or container in which biomolecules (e.g., nucleic acids, polypeptides, polysaccharides, lipids, and the like) are placed; a cell or population of cells; a tissue; an organ; an organism, and the like. Typically the biological system is a cell or population of cells, but the method can also be performed in a vessel using purified or recombinant proteins.

The invention provides siRNA molecules targeted to a transcript encoding any morphogenic protein or morphogenic-related protein. In particular, the invention provides siRNA molecules selectively or specifically targeted to a transcript encoding a polymorphic variant of such a transcript, wherein existence of the polymorphic variant in a subject is indicative of susceptibility to or presence of a musculoskeletal disorder or spondylarthropathic disease. The terms “selectively” or “specifically targeted to”, in this context, are intended to indicate that the siRNA causes greater reduction in expression of the variant than of other variants (i.e., variants whose existence in a subject is not indicative of susceptibility to or presence of a musculoskeletal disorder or spondylarthropathic disease). The siRNA, or collections of siRNAs, can be provided in the form of kits with additional components as appropriate.

Short Hairpin RNA (shRNA)

RNA interference (RNAi), a mechanism of post-transcriptional gene silencing mediated by double-stranded RNA (dsRNA), is useful in a method for treating a musculoskeletal disorder in a mammal by administering to the mammal a nucleic acid molecule (e.g., dsRNA) that hybridizes under stringent conditions to a morphogenic gene or MAP kinase gene, and attenuates expression of said target gene. See Jain, Pharmacogenomics 5: 239-42, 2004 for a review of RNAi and siRNA. A further method of RNA interference in the present invention is the use of short hairpin RNAs (shRNA). A plasmid containing a DNA sequence encoding a particular desired siRNA sequence is delivered into a target cell via transfection or virally mediated infection. Once in the cell, the DNA sequence is continuously transcribed into RNA molecules that loop back on themselves and form hairpin structures through intramolecular base pairing. These hairpin structures, once processed by the cell, are equivalent to transfected siRNA molecules and are used by the cell to mediate RNAi of the desired protein. The use of shRNA has an advantage over siRNA transfection as the former can lead to stable, long-term inhibition of protein expression Inhibition of protein expression by transfected siRNAs is a transient phenomenon that does not occur for times periods longer than several days. In some cases, this can be preferable and desired. In cases where longer periods of protein inhibition are necessary, shRNA mediated inhibition is preferable.

### Full and Partial Length Antisense RNA Transcripts

Antisense RNA transcripts have a base sequence complementary to part or all of any other RNA transcript in the same cell. Such transcripts have been shown to modulate gene expression through a variety of mechanisms including the modulation of RNA splicing, the modulation of RNA transport and the modulation of the translation of mRNA (Denhardt, Ann N Y Acad. Sci. 660: 70, 1992; Nellen, Trends Biochem. Sci. 18: 419, 1993; Baker and Monia, Biochem. Biophys. Acta, 1489: 3, 1999; Xu et al., Gene Therapy 7: 438, 2000; French and Gerdes, Curr. Opin. Microbiol. 3: 159, 2000; Terryn and Rouze, Trends Plant Sci. 5: 1360, 2000).

### Antisense RNA and DNA Oligonucleotides

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but can range from 10 up to approximately 50 nucleotides in length. Binding typically reduces or inhibits the function of the target nucleic acid. For example, antisense oligonucleotides can block transcription when bound to genomic DNA, inhibit translation when bound to mRNA, and/or lead to degradation of the nucleic acid. Reduction in expression of a morphogenic protein or morphogenic polypeptide can be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., 2000, Volumes 313 and 314, Academic Press, San Diego, and references mentioned therein. See also Crooke, S. (ed.) “Antisense Drug Technology: Principles, Strategies, and Applications” (1^{st} Edition) Marcel Dekker; and references cited therein.

Antisense oligonucleotides can be synthesized with a base sequence that is complementary to a portion of any RNA transcript in the cell. Antisense oligonucleotides can modulate gene expression through a variety of mechanisms including modulation of RNA splicing, the modulation of RNA transport, and modulation of the translation of mRNA (Denhardt, Ann N Y Acad. Sci. 660: 70, 1992). Various properties of antisense oligonucleotides including stability, toxicity, tissue distribution, and cellular uptake and binding affinity can be altered through chemical modifications including (i) replacement of the phosphodiester backbone (e.g., peptide nucleic acid, morpholino-oligonucleotides, phosphorothioate oligonucleotides, and phosphoramidite oligonucleotides), (ii) modification of the sugar base (e.g., 2′-O-propylribose and 2′-methoxyethoxyribose), and (iii) modification of the nucleoside (e.g., C-5 propynyl U, C-5 thiazole U, and phenoxazine C) (Wagner, Nat. Medicine 1: 1116, 1995; Varga et al., Immun. Lett. 69: 217, 1999; Neilsen, Curr. Opin. Biotech. 10: 71, 1999; Woolf, Nucleic Acids Res. 18: 1763, 1990).

The invention provides a method of inhibiting expression of a gene encoding a musculoskeletal disorder or spondylarthropathic disease comprising the step of (i) providing a biological system in which expression of a gene encoding a morphogenic protein or MAP kinase protein is to be inhibited; and (ii) contacting the system with an antisense molecule that hybridizes to a transcript encoding the morphogenic molecule or morphogenic protein. According to certain embodiments of the invention the biological system comprises a cell, and the contacting step comprises expressing the antisense molecule in the cell. According to certain embodiments of the invention the biological system comprises a subject, e.g., a mammalian subject such as a mouse or human, and the contacting step comprises administering the antisense molecule to the subject or comprises expressing the antisense molecule in the subject. The expression can be inducible and/or tissue or cell type-specific. The antisense molecule can be an oligonucleotide or a longer nucleic acid molecule. The invention provides such antisense molecules.

### Inhibitory Ribozymes

The invention provides ribozymes capable of inhibiting gene function by targeting mRNA, i.e., destroying mRNA encoding hSMOC polypeptides or polypeptides with morphogenic activity. Thus, RNA and DNA enzymes can be designed to cleave to any RNA molecule, thereby increasing its rate of degradation (Cotten and Birnstiel, EMBO J. 8: 3861-3866, 1989; Usman et al., Nucl. Acids Mol. Biol. 10: 243, 1996; Usman et al., Curr. Opin. Struct. Biol. 1: 527, 1996; Sun et al., Pharmacol. Rev. 52: 325, 2000).

Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme, which is held in close proximity to an enzymatic portion of the RNA that cleaves the target RNA. Thus, the ribozyme recognizes and binds a target RNA through complementary base pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation, or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA to the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

The invention provides a method of inhibiting expression of a gene encoding a morphogenic gene (such as inhibition of hSMOC polypeptide) comprising the step of (i) providing a biological system in which expression of a gene encoding a morphogenic protein is to be inhibited; and (ii) contacting the system with a ribozyme that hybridizes to a transcript encoding the morphogenic molecule or morphogenic protein and directs cleavage of the transcript. According to certain embodiments of the invention the biological system comprises a cell, and the contacting step comprises expressing the ribozyme in the cell. According to certain embodiments of the invention the biological system comprises a subject, e.g., a mammalian subject such as a mouse or human, and the contacting step comprises administering the ribozyme to the subject or comprises expressing the ribozyme in the subject. The expression can be inducible and/or tissue or cell-type specific according to certain embodiments of the invention. The invention provides ribozymes designed to cleave transcripts encoding morphogenic molecules or morphogenic proteins, or polymorphic variants thereof, as described above.

### Inbred Mouse Strains

The invention provides an inbred mouse and an inbred mouse strain that can be generated as described herein and bred by standard techniques, see, e.g., U.S. Pat. Nos. 6,040,495; 5,552,287.

In order to screen for mutations with recessive effects a number of strategies can be used, all involving a further two generations. For example, male G1 mice can be bred to wild-type female mice. The resulting progeny (G2 mice) can be interbred or bred back to the G1 father. The G3 mice that result from these crosses will be homozygotes for mutations in a small number of genes (3-6) in the genome, but the identity of these genes is unknown. With enough G3 mice, a good sampling of the genome should be present.

### Animal Models for Joint Repair

Various animal models have been used for evaluation of possible clinical approaches to joint repair. The most widely used of these include the goat, sheep, and horse, each of which has certain capabilities and limitations (for review, see Reinholz et al., Biomaterials 25: 1511-1521, 2004, this reference is herein incorporated by reference for all purposes; note also detailed information on this subject in connection with the Mar. 3-4, 2005 Meeting of the FDA Cellular, Tissue, and Gene Therapies Advisory committee available on the world wide web at fda.gov/ohrms/dockets/ac/cber05.html#CellularTissueGeneTherapies.

### Therapeutic Applications

The compounds and modulators identified by the methods of the present invention can be used in a variety of methods of treatment. Thus, the present invention provides compositions and methods for treating musculoskeletal disorders including disorders related to bone, muscle, ligaments, tendons, cartilage, and joints. The musculoskeletal disorders can further include spondylarthropathic disease or related diseases. Treatment of a musculoskeletal disease or disorders is within the ambit of regenerative medicine, for example, disorders requiring spinal fixation, spinal stabilization, repair of segmental defects in the body (such as in long bones and flat bones), disorders of the vertebrae and discs including, but not limited to, disruption of the disc annulus such as annular fissures, chronic inflammation of the disc, localized disc herniations with contained or escaped extrusions, and relative instability of the vertebrae surrounding the disc are musculoskeletal disorders. Musculoskeletal disorders also include sprains, strains and tears of ligaments, tendons, muscles, and cartilage; tendonitis, spondylarthropathic disease, tenosynovitis, fibromyalgia, osteoarthritis, rheumatoid arthritis, polymyalgia rheumatica, bursitis, acute and chronic back pain and osteoporosis, sports injuries and work related injuries including sprains, strains and tears of ligaments, tendons, muscles, and cartilage; carpal tunnel syndrome, DeQuervains's disease, trigger finger, tennis elbow, rotator cuff injuries, and ganglion cysts. In addition, musculoskeletal disorders include genetic diseases of the musculoskeletal system such as osteogenesis imperfecta, Duchenne, and other muscular dystrophies. Pain is the most common symptom and is frequently caused by injury or inflammation. Besides pain, other symptoms such as stiffness, tenderness, weakness, and swelling or deformity of affected parts are manifestations of musculoskeletal disorders.

Preferably, treatment using a polypeptide or polynucleotide of the present invention could either be by administering an effective amount of a SMOC polypeptide to the patient, or by removing cells from the patient, supplying the cells with a polynucleotide encoding a SMOC polypeptide, and returning the engineered cells to the patient (ex vivo therapy). Treatment could also be by administering a nucleic acid encoding a SMOC polypeptide, a vector comprising such a nucleic acid, or a host cell expressing a SMOC polypeptide.

### Formulation and Administration of Pharmaceutical Compositions

The nucleic acids, peptides and polypeptides, e.g., hSMOC polypeptide, or a conservatively modified variant, derivative, or analog thereof, can be combined with a pharmaceutically acceptable carrier (excipient) to form a pharmacological composition. Pharmaceutically acceptable carriers can contain a physiologically acceptable compound that acts to, e.g., stabilize, localize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also be used to stabilize or to increase or decrease the absorption of the pharmaceutical composition, including liposomal carriers. The pharmaceutical composition may also be incorporated into biomaterial scaffold or support materials, including those comprised of synthetic polymers, proteins, metals, etc., or combinations thereof. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives that are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of nucleic acids, peptides or polypeptides e.g., hSMOC polypeptide, or a conservatively modified variant, derivative, or analog thereof, are dissolved in a pharmaceutically acceptable carrier, e.g., an aqueous carrier if the composition is water-soluble. Examples of aqueous solutions that can be used in formulations for enteral, parenteral, or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents or stabilizers. For example, the solution can contain sodium acetate, sodium lactate, sodium chloride, potassium chloride, calcium chloride, sorbitan monolaurate, or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders, or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable, or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, or ethanol.

Nucleic acids, peptides or polypeptides, e.g., hSMOC polypeptide, or a conservatively modified variant, derivative, or analog thereof—when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. (See, e.g., Sayani, Crit. Rev. Ther. Drug Carrier Syst. 13: 85-184, 1996.) For topical, transdermal administration, the agents are formulated into ointments, creams, salves, powders and gels. Transdermal delivery systems can also include, e.g., patches.

The nucleic acids, peptides, or polypeptides of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention. (See, e.g., Putney, Nat. Biotechnol. 16: 153-157, 1998).

For inhalation, the nucleic acids, peptides or polypeptides of the invention can be delivered using any system known in the art, including dry powder aerosols, liquid delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The nucleic acids, peptides or polypeptides of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, or nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS. (See e.g., Gurun, Anesth Analg. 85: 317-323, 1997). For example, intra-carotid artery injection is preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's. (See also, Bai, J. Neuroimmunol. 80: 65-75, 1997; Warren, J. Neurol. Sci. 152: 31-38, 1997; Tonegawa, J. Exp. Med. 186: 507-515, 1997.)

In one aspect, the pharmaceutical formulations comprising nucleic acids, peptides or polypeptides, e.g., hSMOC polypeptide, or a conservatively modified variant, derivative, or analog thereof, are incorporated in lipid monolayers or bilayers, e.g., liposomes, see, e.g., U.S. Pat. Nos. 6,110,490; 6,096,716; 5,283,185; 5,279,833. The invention also provides formulations in which water-soluble nucleic acids, peptides or polypeptides of the invention have been attached to the surface of the monolayer or bilayer. For example, peptides can be attached to hydrazide-PEG-(distearoylphosphatidyl)ethanolamine-containing liposomes. (See, e.g., Zalipsky Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides, and/or polypeptides of the invention are incorporated within micelles and/or liposomes. (See, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art. (See, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4, 235,871, 4,501,728 and 4,837,028.)

The pharmaceutical compositions are generally formulated as sterile, substantially isotonic, and in full compliance with all Good Manufacturing Practice (GMP) regulations of the U.S. Food and Drug Administration.

### Treatment Regimens and Phamacokinetics

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, and the like. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer Science 249: 1527-1533, 1990.

In therapeutic applications, compositions are administered to a patient suffering from a musculoskeletal disorder or spondylarthropathic disease to at least partially arrest the condition or a disease and/or its complications. For example, in one aspect, a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF) or a joint space or structure.

The invention provides pharmaceutical compositions comprising one or a combination of therapeutic proteins, such as, for example, a SMOC polypeptide, or a fragment or conservatively modified variant, derivative, or analog thereof; or one or more nucleic acid molecules comprising, for example, a nucleic acid sequence that encode a SMOC polypeptide or a biologically active fragment or conservatively modified variant of a SMOC polypeptide, or a nucleic acid that reduces or inhibits the expression of a SMOC polypeptide, such as, for example, an antisense oligonucleotide, a double stranded RNA oligonucleotide (RNAi), or a DNA oligonucleotide containing a nucleotide sequence encoding a shRNA molecule, formulated together with a pharmaceutically acceptable carrier. Some compositions include a combination of multiple (e.g., two or more) therapeutic proteins, e.g., a hSMOC polypeptide, or a conservatively modified variant, derivative, or analog thereof, by itself or in combination with other therapeutic agents, such as, for example, one or more additional BMP antagonists. For example, it is well-known in the art that interruption of a metabolic or signaling pathway at two distinct points, such as is done with combination antimicrobial or anticancer therapy, is likely to produce a synergistic effect. In turn, it is often the case that therapeutic effectiveness can be enhanced and side effects reduced by this approach.

In prophylactic applications, pharmaceutical compositions or medicaments are administered to a patient susceptible to, or otherwise at risk of a disease or condition (e.g., a musculoskeletal disorder or spondylarthropathic disease) in an amount sufficient to eliminate or reduce the risk, lessen the severity, or delay the outset of the disease, including biochemical, histologic and/or behavioral symptoms of the disease, its complications, and intermediate pathological manifestations presenting during development of the disease. In therapeutic applications, compositions or medicants are administered to a patient suspected of, or already suffering from such a disease in an amount sufficient to cure, or at least partially arrest, the symptoms of the disease (biochemical, histologic, and/or behavioral), including its complications and intermediate pathological manifestations in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient BMP antagonist response, immune response, or other desired response has been achieved. Typically, any response is monitored and repeated dosages are given if the response starts to wane.

### Effective Dosages

Effective doses of the therapeutic proteins, such as, for example, a SMOC polypeptide, or a fragment or conservatively modified variant, derivative, or analog thereof; or one or more of the nucleic acid molecules comprising, for example, a nucleic acid sequence that encodes a SMOC polypeptide or a biologically active fragment or conservatively modified variant of a SMOC polypeptide, or a nucleic acid that reduces or inhibits the expression of a SMOC polypeptide, such as, for example, an antisense oligonucleotide, a double stranded RNA oligonucleotide (RNAi), or a DNA oligonucleotide containing a nucleotide sequence encoding a shRNA molecule, for the treatment of a musculoskeletal disorder or spondylarthropathic disease described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Doses need to be titrated to optimize safety and efficacy.

For administration with a polypeptide, peptidomimetic, or nucleic acid composition, the dose ranges from about 0.0001 to 100 mg/kg, and more usually 0.01 to 5 mg/kg, of the host body weight. For example doses can be 1 mg/kg body weight or 10 mg/kg body weight or within the range of 1-10 mg/kg. An exemplary treatment regime entails administration once per every two weeks or once a month or once every 3 to 6 months. In some methods, two or more polypeptide, peptidomimetic, or nucleic acid compositions with different specificities are administered simultaneously, in which case the dose of each polypeptide, peptidomimetic, or nucleic acid composition administered falls within the ranges indicated. Polypeptide, peptidomimetic, or nucleic acid composition is usually administered on multiple occasions. Intervals between single dosages can be weekly, monthly or yearly. Intervals can also be irregular, as indicated by measuring blood levels of polypeptide, peptidomimetic, or nucleic acid composition in the patient, or other appropriate indicators of the composition's pharmacologic disposition. In some methods, dose is adjusted to achieve a plasma polypeptide or nucleic acid composition concentration of 1-1000 μg/ml and in some methods 25-300 μg/ml. Alternatively, polypeptide or nucleic acid composition can be administered as a sustained release formulation, in which case less frequent administration is required. Dose and frequency vary depending on the half-life of the polypeptide or nucleic acid composition in the patient. In general, human polypeptide compositions show the longest half-life, followed by chimeric polypeptide compositions, and nonhuman polypeptide compositions. The dose and frequency of administration can vary depending on whether the treatment is prophylactic or therapeutic. In prophylactic applications, a relatively low dose is administered at relatively infrequent intervals over a long period of time. Some patients continue to receive treatment for the rest of their lives. In therapeutic applications, a relatively high dose at relatively short intervals is sometimes required until progression of the disease is reduced or terminated, and preferably until the patient shows partial or complete amelioration of symptoms of disease. Thereafter, the patient can be administered a prophylactic regime.

Doses for nucleic acids range from about 10 ng to 1 g, 100 ng to 100 mg, 1 μg to 10 mg, or 30-300 μg DNA per patient. Doses for infectious viral vectors vary from 10-100, or more, virions per dose.

### Routes of Administration

Polypeptide or peptidomimetic compositions for inducing a therapeutic response, such as, for example, a SMOC polypeptide, or a fragment or conservatively modified variant, derivative, or analog thereof; or compositions comprising one or more nucleic acid molecules comprising, for example, a nucleic acid sequence that encode a SMOC polypeptide or a biologically active fragment or conservatively modified variant of a SMOC polypeptide, or a nucleic acid that reduces or inhibits the expression of a SMOC polypeptide, such as, for example, an antisense oligonucleotide, a double stranded RNA oligonucleotide (RNAi), or a DNA oligonucleotide containing a nucleotide sequence encoding a shRNA molecule, for the treatment of a musculoskeletal disorder or spondylarthropathic disease, described herein, can be administered by parenteral, topical, intravenous, oral, subcutaneous, intraarterial, intracranial, intraperitoneal, intranasal, or intramuscular means for prophylaxis as inhalants for antibody preparations and/or therapeutic treatment. The most typical route of administration of a therapeutic peptide or peptidomimetic agent is subcutaneous, although other routes can be equally effective. The next most common route is intramuscular injection. This type of injection is most typically performed in the arm, shoulder, or leg muscles. In some methods, agents are injected directly into a particular tissue, for example intracranial injection or convection-enhanced delivery. Intramuscular injection or intravenous infusion are preferred for administration of antibody. In some methods, particular therapeutic peptide or peptidomimetic composition are delivered directly into the cranium, a joint or joint-associated structure, or other anatomic location. In some methods, therapeutic peptide or peptidomimetic composition are administered as a sustained release composition or device, such as a Medipad™ device.

Agents of the invention can optionally be administered in combination with other agents that are at least partly effective in treating various musculoskeletal disorders or spondylarthropathic disease.

### Formulation

Polypeptide, peptidomimetic, or nucleic acid compositions for inducing a response to morphogenic gene products comprising therapeutic proteins, such as, for example, a SMOC polypeptide, or a fragment or conservatively modified variant, derivative, or analog thereof; or comprising one or more nucleic acid molecules comprising, for example, a nucleic acid sequence that encode a SMOC polypeptide or a biologically active fragment or conservatively modified variant of a SMOC polypeptide, or a nucleic acid that reduces or inhibits the expression of a SMOC polypeptide, such as, for example, an antisense oligonucleotide, a double stranded RNA oligonucleotide (RNAi), or a DNA oligonucleotide containing a nucleotide sequence encoding a shRNA molecule, for the treatment of a musculoskeletal disorder or spondylarthropathic disease described herein, are often administered as pharmaceutical compositions comprising an active therapeutic agent, i.e., and a variety of other pharmaceutically acceptable components. See the most recent edition of Remington's Pharmaceutical Science (e.g., 20^{th} ed., Mack Publishing Company, Easton, Pa., 2000). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances, and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990; Hanes, Advanced Drug Delivery Reviews 28: 97-119, 1997. The agents of this invention can be administered in the form of a depot injection or implant preparation, which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations, or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (see Glenn, Nature 391: 851, 1998). Co-administration can be achieved by using the components as a mixture or as linked molecules obtained by chemical crosslinking or expression as a fusion protein.

Alternatively, transdermal delivery can be achieved using a skin patch or using transferosomes. Paul, Eur. J. Immunol. 25: 3521-24, 1995; Cevc, Biochem. Biophys. Acta 1368: 201-15, 1998.

The pharmaceutical compositions are generally formulated as sterile, substantially isotonic, and in full compliance with all Good Manufacturing Practice (GMP) regulations of the U.S. Food and Drug Administration.

### Toxicity

Preferably, a therapeutically effective dose of the polypeptide or peptidomimetic compositions or nucleic acid compositions comprising, for example, a SMOC polypeptide, or a fragment or conservatively modified variant, derivative, or analog thereof; or one or more nucleic acid molecules comprising, for example, a nucleic acid sequence that encode a SMOC polypeptide or a biologically active fragment or conservatively modified variant of a SMOC polypeptide, or a nucleic acid that reduces or inhibits the expression of a SMOC polypeptide, such as, for example, an antisense oligonucleotide, a double stranded RNA oligonucleotide (RNAi), or a DNA oligonucleotide containing a nucleotide sequence encoding a shRNA molecule, described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in estimating a dosage range that is not toxic for use in human. The dosage of the proteins described herein lies preferably within a range of concentrations that include the effective dose with little or no toxicity. The dosage can vary within this range depending upon the dosage form employed and the route of administration utilized. The exact formulation, route of administration, and dosage can be chosen by the individual physician in view of the patient's condition. (See, e.g., Hardman, J. G. L. E. Limbird, and A. G. Gilman, 2001, THE PHARMACOLOGICAL BASIS OF THERAPEUTICS (McGraw-Hill Professional Publishers).

### Kits

For use in diagnostic, research, and therapeutic applications suggested above, kits are also provided by the invention. In the diagnostic and research applications such kits can include any or all of the following: assay reagents; buffers; SMOC antibodies; proteins, such as, for example, a SMOC polypeptide, or a fragment or conservatively modified variant, derivative, or analog thereof; one or more nucleic acid molecules comprising, for example, a nucleic acid sequence that encode a SMOC polypeptide or a biologically active fragment or conservatively modified variant of a SMOC polypeptide; a nucleic acid that reduces or inhibits the expression of a SMOC polypeptide, such as, for example, an antisense oligonucleotide, a double stranded RNA oligonucleotide (RNAi), or a DNA oligonucleotide containing a nucleotide sequence encoding a shRNA molecule; hybridization probes and/or primers; PCR primers; ribozymes; dominant negative hSMOC variant polypeptides or polynucleotides; small molecule inhibitors or activators of BMP or BMP variants, and the like. A therapeutic product can include sterile saline or another pharmaceutically acceptable emulsion and suspension base as described above.

Accordingly, kits of the present invention can contain any reagents that specifically hybridize to hSMOC variant nucleic acids, e.g., hSMOC variant probes and primers, and hSMOC-specific reagents that specifically bind to and/or modulate the activity of a hSMOC variant protein, e.g., hSMOC variant antibodies, hSMOC variant ligands, or other compounds, that are used to treat hSMOC-associated or BMP-associated diseases or conditions. Kits of the present invention can also contain additional agents that can be administered concomitantly with the compounds of the present invention. In addition, kits can contain reagents or other components used to locate hSMOC, BMP, or MAP kinase polypeptides, or nucleic acid probes, primers, or other materials that can be used to detect biological activation of MAP kinase polypeptides. These may include, but are not limited to, specific antibodies or antisera, e.g., to MAP kinase or BMP proteins associated with activation of the polyp eptides of the invention and/or PCR primers to detect genes transcribed in response to BMP signaling.

In addition, the kits can include instructional materials containing directions (i.e., protocols) for the practice of the methods of this invention. While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The invention will be further described with reference to the following examples; however, it is to be understood that the invention is not limited to such examples.

The following examples of specific aspects for carrying out the present invention are offered for illustrative purposes only, and are not intended to limit the scope of the present invention in any way.

### Exemplary Aspects

### EXAMPLE 1

### Isolation and Characterization of Xenopus SMOC

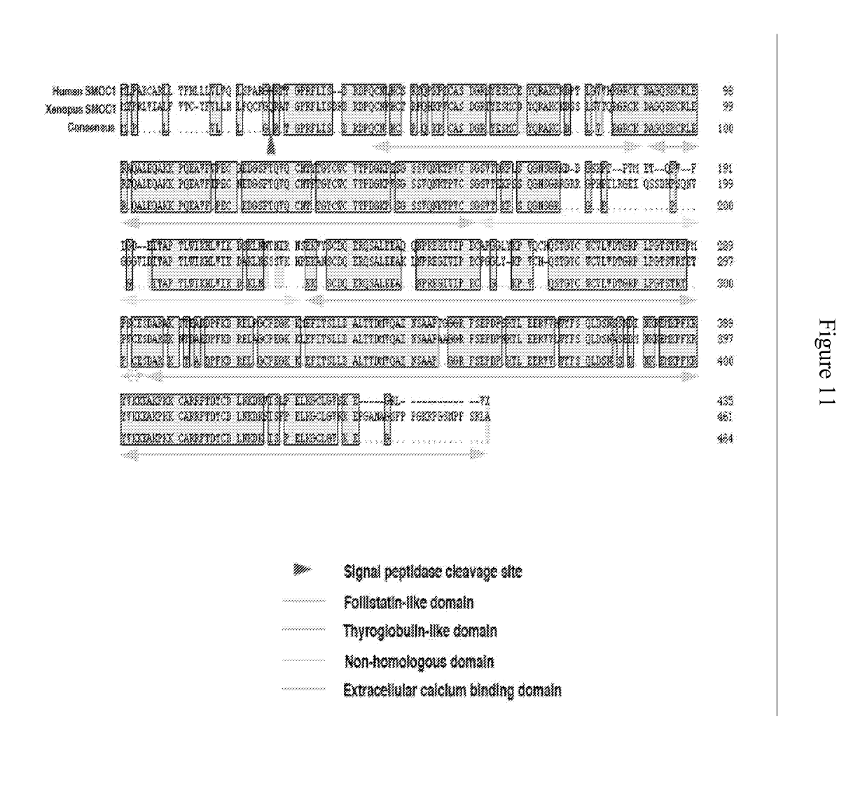

Though mammals have two forms of SMOC, extensive attempts to isolate more than one form from Xenopus were unsuccessful and searches of Xenopus (laevis and tropicalis) EST databases and the Joint Genome Institute (JGI) Xenopus Tropicalis genomic database (version 4.1) revealed only a single form. The Xenopus SMOC open reading frame is 74% and 50% identical to human SMOC-1 and SMOC-2, respectively. Therefore, the gene product is most likely the Xenopus ortholog of human SMOC-1. XSMOC-1 is composed of 463 amino acids, compared to 434 in human SMOC-1. The difference is due largely to an additional 19 amino acids at the C-terminal end and an additional 9 amino acids within a domain that lacks homology to other proteins (termed the non-homologous domain). The domain structure of XSMOC-1 and mammalian SMOC1/2 is conserved (See FIG. 11). XSMOC-1 has a 25 amino acid leader sequence followed by a predicted signal peptidase cleavage site between amino acid 25 and 26 (CFG-R). The identities between human SMOC-1 and XSMOC-1 within the conserved domains of the mature protein are as follows: Follistatin-like domain—72%; Thyroglobulin-like domain 1—93%; non-homologous domain—42%; Thyroglobulin-like domain 2—79%; Calcium-binding domain—88%.

XSMOC-1 first became detectable by RT-PCR at stage 12.5, corresponding to late gastrulation/early neurulation, and remained at consistent levels throughout neurula and tailbud stages (FIG. 1A). Hybridization in situ in whole embryos showed XSMOC-1 to be expressed initially at stage 12.5 at the anterior of the embryo with a dorso-ventral distribution (FIG. 1B). At stage 14, XSMOC-1 was localized within the anterior-ventral region and also lateral to the developing neural plate (FIG. 1C). This staining pattern continued throughout neurulation (FIG. 1C-E). At later stages (20 to 25), XSMOC-1 was localized dorsal to the cement gland, the ventral region of the developing eye FIG. 1H), and the developing pronephros (FIG. 1F-J). Expression was also observed in the mesencephalon and rhombencephalon (FIG. 1I, J) with prolonged color development. By stage 30, XSMOC-1 was also observed in the pharyngeal arches (not shown). Transverse sections of overstained embryos at stage 25 confirmed the ventral eye expression domain (FIG. 1K) and revealed XSMOC-1 to be localized to the lateral regions of the mid- (not shown) and hind-brain (FIG. 1L). In the trunk, expression was observed throughout the pronephros and in subepithelial neural crest cells migrating laterally to the somites (FIG. 1M).

### EXAMPLE 2

### Gain-of-Function of XSMOC-1 Produces a Phenotype and Molecular Marker Pattern Consistent with Action as a BMP Antagonist

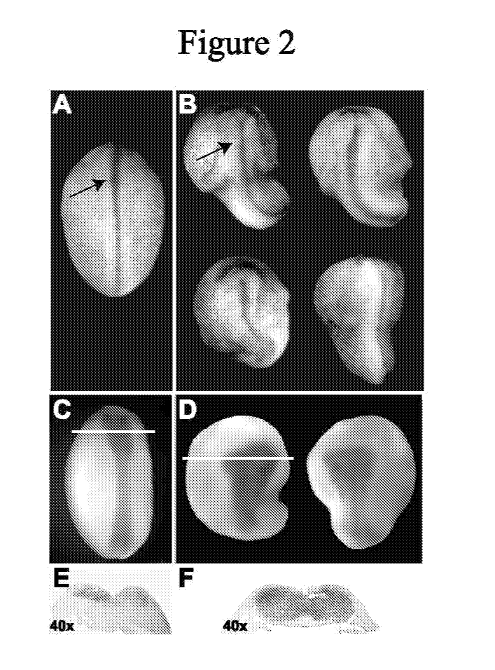

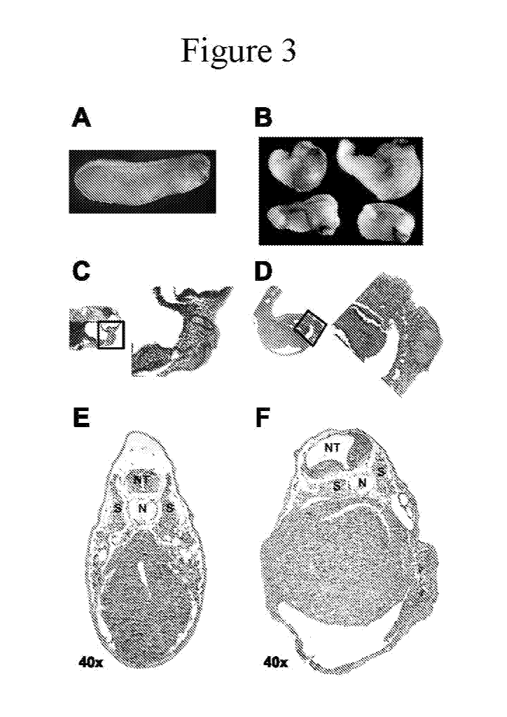

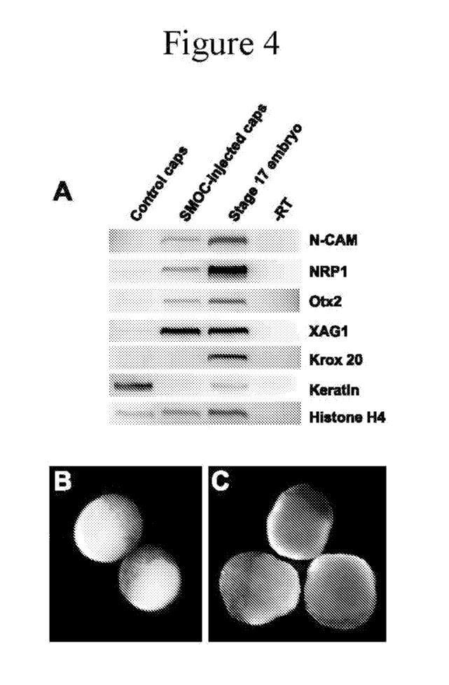

Bilateral injection of mRNA (300 pg) encoding XSMOC-1 (FIGS. 2 and 3) or Zebrafish SMOC-2 (not shown) at the two-cell stage produced exaggerated dorsal/anterior structures, most prominently enlarged heads and cement glands. The phenotype was apparent at stage 17 (FIG. 2B). Whole mount hybridization in situ analysis of Sox2 expression demonstrated that relative to controls (FIG. 2C) the neural plate was expanded in the dorsalized embryos (FIG. 2D). Transverse sections taken through the anterior region of overstained embryos showed that, unlike controls (FIG. 2E), Sox2 expression occupied the majority of the tissue dorsal to the archenteron roof in XSMOC-1 overexpressing embryos (FIG. 2F). By stage 26 the dorsalization was more apparent (FIG. 3B) and histological analysis of sagittal sections revealed grossly hypertrophied columnar epithelium in the cement gland (FIG. 3D). Transverse sections through stage 33 XSMOC-1 overexpressing embryos showed enlargement of the neural tube and disorganized somites (FIG. 3F). Animal cap explants from embryos injected with XSMOC-1 mRNA expressed anterior neuroectodermal (Otx2 and XAG-1) and panneural (NCAM and NRP-1) markers, but not the posterior neural marker Krox-20 (FIG. 4A). In addition, the epithelial marker keratin was down-regulated (FIG. 4A), supporting conversion from epithelial to neural cell fate. The biological effects of XSMOC-1 overexpression in these assays were consistent with that of a Bone Morphogenetic Protein (BMP) antagonist (20,21).

To examine whether XSMOC-1 action was cell-autonomous or was effective away from its point of origin, we assayed conjugated animal caps by whole mount hybridization in situ. Animal caps from wild-type embryos injected with control or XSMOC-1 mRNAs were conjugated to non-injected albino animal caps and analyzed by hybridization in situ for the anterior neuroectodermal marker Otx2 when sibling embryos reached stage 17. Otx2 was not detectable in the controls (FIG. 4B), but was readily detectable in the albino non-injected caps conjugated to XSMOC-1 expressing wild-type caps (FIG. 4C), indicating that XSMOC-1 can act at a distance from its cellular origin.

### EXAMPLE 3

### Loss-of Function of XSMOC-1 Arrests Development at Neurulation

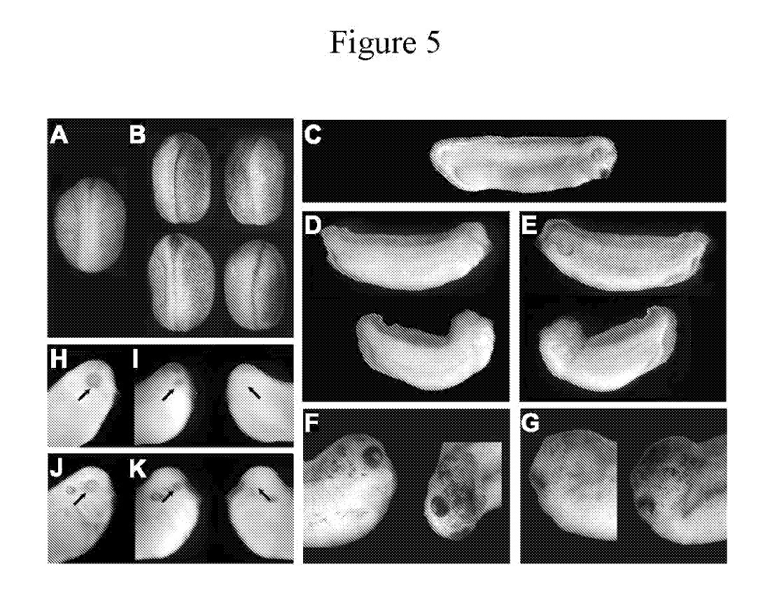

Injection of Xenopus embryos with morpholino antisense oligonucleotides has been used widely and effectively to study the effects of blocking the synthesis of selected proteins (See gene-tools.com; (22)). An antisense morpholino to XSMOC-1 (XSMOC-1 MO) located at position −20 to +5 was designed to examine the effect of down-regulation of XSMOC-1 during early Xenopus development. Initial studies were conducted on embryos injected unilaterally with 6 ng of XSMOC-MO at the two-cell stage (FIG. 5). At stage 17 mild abnormalities were observed in the developing neural axis (FIG. 5B). By stage 32, compared to controls (FIG. 5C), anterior defects (mild ventralization) were apparent (FIG. 5D, E) and eye and other anterior structures were absent or severely dysmorphic on the injected side; corresponding structures on the non-injected side were also affected, but less severely (FIG. 5D, E). At stage 38, these differences were more obvious (FIG. 5G). Whole mount hybridization in situ studies of stage 32 embryos for Otx2 (FIG. 5H, I) and Tbx2 (FIG. 5J, K) revealed aberrant expression of these markers in the eye field on the XSMOC-1 MO-injected side (FIG. 5I, K right panels). Otx2 expression was diminished in the developing eye field on the non-injected side and was completely absent on the MO-injected side (FIG. 5I). Expression of Tbx2 on the non-injected side was similar to controls (FIG. 5J; K, left side), but expression in the eye field was diminished (FIG. 5K, left side). On the MO-injected side, Tbx2 expression was absent from the eye region and branchial arches, but was present in the cranial ganglia, otic vesicle, and frontonasal process (FIG. 5K, right side).

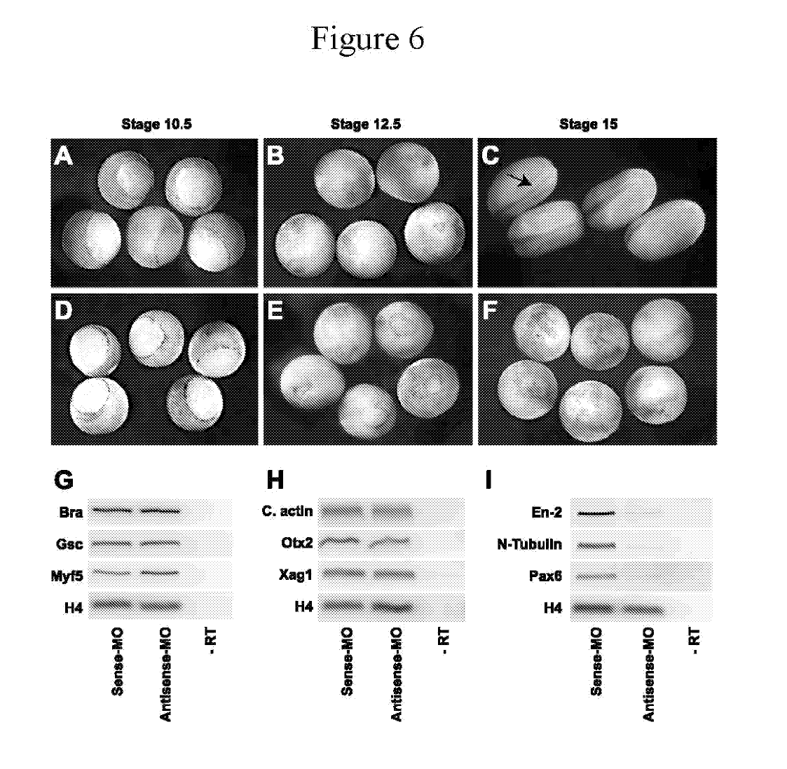

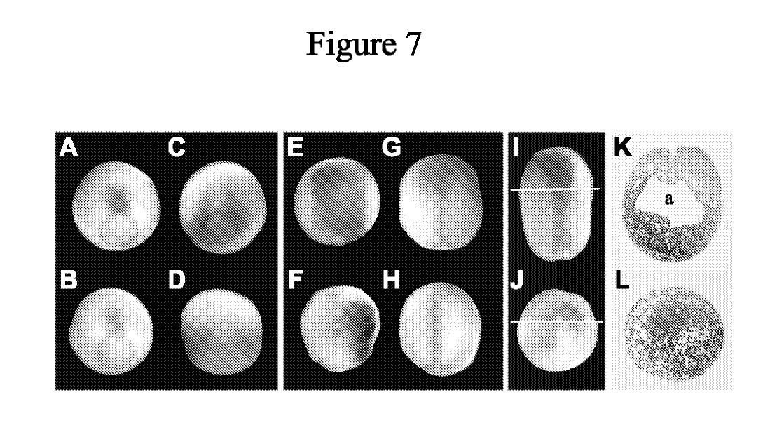

Bilateral injections of 6 ng of XSMOC-MO at the two-cell stage resulted in complete developmental arrest at the end of gastrulation (FIG. 6). Development appeared normal until late gastrulation (FIG. 6D and E), and RT-PCR analyses revealed normal expression of the markers Brachyury, Goosecoid, and Myf-5 at stage 10.5 (FIG. 6G) and of cardiac actin, Otx2, and XAG at stage 12 (FIG. 6H). Developmental arrest immediately prior to neurulation appeared to be very abrupt and near total (FIG. 6F); the post-gastrulation markers En-2, Pax6, and N-Tubulin were expressed only weakly (FIG. 6I). Hybridization in situ analyses at stages 11 to 11.5 demonstrated some disturbance of the normal expression patterns of the organizer and presumptive notochord marker XNot and the myogenic marker myf5 in XSMOC-1 MO-injected embryos (FIG. 7A-D). At stage 12.5, XNot expression in the presumptive notochord of XSMOC-1 MO-injected embryos was abnormal, and the neural plate marker XSox2 was disturbed severely (FIG. 7E-H). At stage 15, convergent extension associated with neurulation failed to occur in XSMOC-1 MO-injected embryos, and the XSox2 expression pattern was disrupted further (FIG. 7I, J). Histological analysis of these embryos revealed the absence of the archenteron and any recognizable dorsal structures (FIG. 7L). These findings suggest that the effects XSMOC-1 loss-of-function are specific to one or more processes occurring near the end of gastrulation and are not due to disruption of a more global process necessary for cell viability.

The specificity of the XSMOC-1 morpholino effect on Xenopus embryos was confirmed as follows: Co-injection of XSMOC-1 MO (l2ng) with Zebrafish SMOC-2 mRNA (600pg), which cannot hybridize to XSMOC-1 MO, produced partial to full rescue of bilaterally-injected embryos. Injection of a second non-overlapping XSMOC-1 antisense MO located at position −39 to −63 (XSMOC-1 MO2) produced the same phenotype as XSMOC-1 MO (65% of embryos arrested prior to neurulation in three separate experiments; n=96) in bilaterally-injected embryos, at a dose of 30 ng per blastomere at the two cell stage (not shown).

### EXAMPLE 4

### XSMOC-1 Blocks the Effects of BMP2 but not Activin and Acts Downstream of the BMPR1B-Receptor

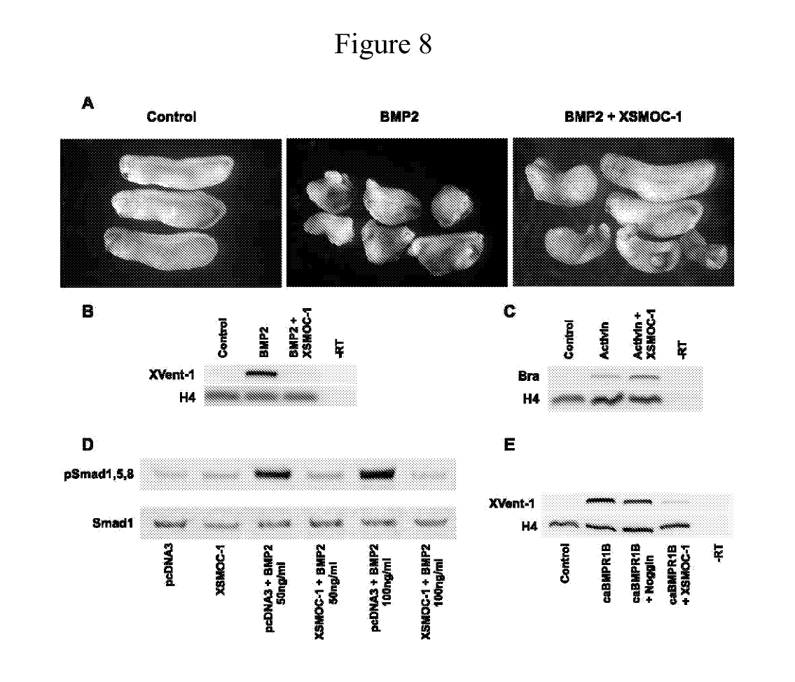

Since overexpression of XSMOC-1 in Xenopus embryos produced a phenotype similar to that observed for BMP antagonists, we analyzed the effect of XSMOC-1 on BMP2 and Activin activity. Both are members of the TGF-□ superfamily, but signal via different serine-threonine kinase receptors. Over expression of BMP2 in Xenopus embryos produced a strongly ventralized phenotype ((23); FIG. 8A) that could be rescued partially or completely by co-expression of XSMOC-1 (FIG. 8A). RT-PCR analysis demonstrated that BMP2-mediated induction of the ventral marker XVent-1 was blocked completely by co-expression of XSMOC-1 in animal cap explants (FIG. 8B). In contrast, induction of Brachyury by Activin was not inhibited by XSMOC-1 (FIG. 8C) Inhibition of BMP2 activity by XSMOC-1 was also demonstrated in mammalian cell culture (FIG. 8D). Mouse 3T3 fibroblasts were chosen as they have been shown previously to respond to exogenous BMP2/4/7 (24). Cells were transiently transfected with pcDNA3 or pcDNA3-XSMOC-1 and incubated in the presence or absence of recombinant human BMP2 at 50 or 100 ng/ml for one hour. Analysis of cell lysates demonstrated that induction of phospho-Smad 1, 5, or 8 was inhibited by XSMOC-1 at both concentrations of BMP2 (FIG. 8D).

To investigate whether XSMOC-1 acts by direct binding to ligand, we studied its effect in the presence of the constitutively active chicken BMP receptorl B (caBMPR1B). Overexpression of caBMPR1B has been shown to promote signaling of the BMP2/4/7 family in the absence of bound ligand (25) and, consistent with this expectation, animal cap explants from Xenopus embryos injected with caBMPR1B mRNA expressed the ventral marker XVent-1 (FIG. 8E). As expected, the BMP antagonist noggin, which acts extracellularly by direct ligand binding, did not reverse this effect (FIG. 8E). However, expression of XVent-1 in caps from embryos injected with both XSMOC-1 and caBMPRIB mRNA was expressed only weakly (FIG. 8E), indicating that XSMOC-1 does not inhibit BMP signaling via direct binding to BMPs. It also suggests that XSMOC-1 acts downstream of the BMP receptor.

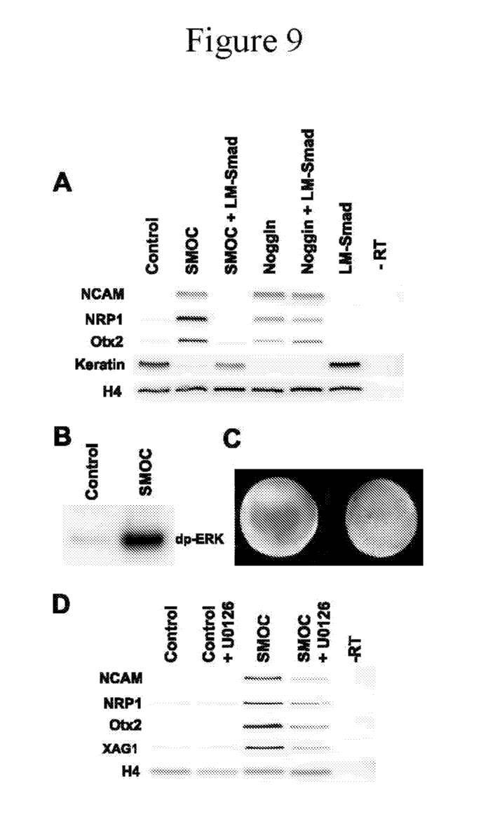

BMP receptors signal through C-terminal phosphorylation of Smad (for review see (26)). This can be inhibited by activation of the MAP Kinase/ERK pathway, which results in Smad phosphorylation within the linker region, effectively blocking C-terminal phosphorylation (27-29). To evaluate the possibility that XSMOC-1 acts via this mechanism, we studied the effect of XSMOC-1 in the presence of linker mutant Smad1 (LM-Smad1). LM-Smadl has four serine-to-alanine substitutions at conserved PXSP sites (also present in Smad 5 and 8) that cannot be phosphorylated by dp-ERK (26) and lacks BMP inhibitory activity. Animal caps from injected embryos were analyzed by RT-PCR for a number of anterior markers (FIG. 9A). In the presence of LM-Smad1, XSMOC-1 did not induce the synthesis of the neural markers N-CAM, Otx2, or NRP-1. In contrast, noggin, which acts by direct binding to BMPs, continued to induce these markers in the presence of LM-Smad-1 (FIG. 9A). In accordance with LM-Smad-1 inhibiting the activity of XSMOC-1, the epidermal marker, keratin, was expressed in control caps, and in the presence of LM-Smad1 or LM-Smad1 plus X-SMOC-1 (FIG. 9A). Keratin was not detected in the presence of XSMOC-1 alone, noggin, or noggin plus LM-Smad1 (FIG. 9A). Further evidence that XSMOC-1 acts through the MAP kinase signaling pathway was obtained by comparing ERK phosphorylation in control and XSMOC-1 loaded animal caps using an antibody specific for the activated diphospho form of this MAP kinase (dp-ERK). Dp-ERK is the kinase responsible for linker phosphorylation of Smadl, 5, and 8 (27,28), and XSMOC-1 overexpression was associated with markedly increased levels of dp-ERK (FIG. 9B). Conversely, in stage 12.5 XSMOC-1 morpholino-injected embryos, dp-ERK activity was absent in the anterior region of the embryo (FIG. 9C).

Dp-ERK formation can be inhibited by the chemical inhibitor U0126, which blocks the activity of MAPK/ERK kinase (MEK; (30)). Animal caps from XSMOC-1-injected embryos were incubated in the presence or absence of U0126 (50 mM) until control embryos reached stage 17. RT-PCR analysis of anterior neuroectodermal (Otx2 and XAG-1) and panneural (NCAM and NRP-1) markers demonstrated that in the presence of U0126 there was a marked reduction in XSMOC-1 activity (FIG. 9D).

### EXAMPLE 5

### XSMOC-1 Inhibits BMP Signaling Downstream of Receptor Binding and is a Required Protein for Xenopus Neurulation

Previous studies on mammalian SMOC in adult tissues identified two closely related genes, SMOC-1 and -2, which have been characterized as extracellular calcium-binding proteins (4,5) with angiogenic and growth factor-potentiating activities (6). Unlike mammals, the Xenopus genome appears to contain only one SMOC gene, the ortholog of mammalian SMOC-1. The domain structure of XSMOC-1 and mammalian SMOC1/2 is conserved and there is a high degree of identity within each of the domains, with the exception of the region exhibiting no homology to other proteins (See FIG. 11). We observed XSMOC-1 to be a zygotic transcript initially expressed at the anterior of the embryo at the end of gastrulation and onset of neurulation (FIG. 1). In neurula embryos XSMOC-1 was expressed lateral to the developing neural plate (FIG. 1 C) and at the early tail bud stage was present in the early pronephric anlage (FIG. 1F). In addition to the pronephric expression, later tail bud embryos expressed XSMOC-1 in the ventral region of the developing eye (FIG. 1H, K), the lateral aspects of the mid- and hindbrain (FIG. 1 1, J, L), and trunk neural crest cells passing laterally to the somites (FIG. 1M). To examine SMOC function during embryological development, we used various assays in the Xenopus model system.

Overexpression of XSMOC-1 in Xenopus embryos produced a dorsalized phenotype and pattern of marker induction suggestive of a BMP antagonist (19,20). Similar to the BMP antagonists noggin and chordin, XSMOC-1 induced anterior (Otx2, Nrp-1, and XAG), but not posterior (Krox 20) neural markers (FIG. 3). Co-expression experiments in Xenopus revealed that XSMOC-1 was able to inhibit the activity of BMP2, which signals through Smadl, 5, or 8, (31) but not Activin, which signals through Smad2 or 3 (FIG. 8) Inhibition of BMP2 signaling by XSMOC-lwas also demonstrated in mouse 3T3 fibroblasts (FIG. 8D). Unlike noggin and chordin, which are first expressed in the Spemann organizer near the onset of gastrulation, XSMOC-1 was not expressed until the end of gastrulation (stage 12.5) and at the pole opposite to the organizer (FIG. 1B). This pattern is consistent with a developmental role for XSMOC-1 in processes initiated following the onset of gastrulation. At later stages (20-26), XSMOC-1 expression in the developing pronephros (FIG. 1F-J and M) and the ventral region of the developing eye (FIG. 1H and K) suggests a possible role in the organogenesis of these structures. Potential targets for the BMP antagonist activity of XSMOC-1 would be BMP7 in the pronephros (31,32), and BMP4, BMP7, and GDF6 in the developing eye (33-35).

Of the many BMP antagonists described to-date, including noggin, chordin, follistatin, cerberus, dan, and gremlin (for review see (1)), most act by direct interaction with BMP ligands to prevent receptor binding or activation. To test whether XSMOC-1 were acting by a similar mechanism, we used a constitutively active type I BMP serine/threonine kinase receptor (caBMPR1B), which activates BMP2/4/7 signaling even in the absence of ligand (25,36). In the presence of caBMPR1B, noggin did not induce the expression of anterior neural markers in animal cap assays (FIG. 8E), consistent with expectation. If XSMOC-1 were acting by a similar mechanism, it would also be expected to be ineffective in the presence of the constitutively active receptor. This was not the case; XSMOC-1 continued to induce expression of anterior neural markers when co-expressed with caBMPR1B (FIG. 8E). The mechanism by which extracellular XSMOC-1 acts as a BMP antagonist appears not to be primarily via direct binding to BMPs, but at a point downstream of the receptor.

Activated BMP receptor serine/threonine kinases phosphorylate intracellular Smads (R-Smads) at C-terminal serine residues, resulting in their translocation to the nucleus to form transcriptional complexes (For review see (26)). An alternative mechanism for interfering with BMP signaling is via activation of the mitogen-activated protein kinase (MAPK) pathway upon ligand (e.g., epidermal growth factor—EGF, fibroblast growth factor—FGF, or insulin-like growth factor—IGF) binding to tyrosine kinases (27-29). The resulting intracellular phosphorylation of the MAP kinase, extracellular signal-regulated kinase (ERK), produces diphospho-ERK (dp-ERK). This, in turn, phosphorylates Smad1, 5, and 8 on serine residues at four conserved PXSP sites within the linker region (27,28). As a consequence, linker-phosphorylated Smad is bound by the ubiquitin ligase Smurf1, resulting in polyubiquitinization and proteasome-dependent degradation in addition to inhibition of Smad nuclear translocation (29). This sequence of events leads to an inhibition of BMP signal transduction. It has been shown that a mutant form of Smad1 (LM-Smad1), which cannot be phosphorylated within the linker region, is unable to inhibit BMP activity (27). When LM-Smad1 was overexpressed in Xenopus embryos, XSMOC-1 activity was lost (FIG. 9A), indicating that XSMOC-1 elicits its effect on BMP signaling by inducing linker-phosphorylation of Smad1, 5, or 8. If this is correct, then one might expect there to be an elevation in dp-ERK levels in response to X-SMOC1 overexpression. This was the case; immunoblot analysis of animal cap explants overexpressing XSMOC-1 demonstrated a dramatic increase in the level of dp-ERK (FIG. 9B). Further support for XSMOC-1 acting via the MAPK pathway came from studies using the MAPK/ERK kinase (MEK) inhibitor U0126 (30). In the presence of U0126, XSMOC-1 activity, as measured by its ability to induce neural markers, was markedly reduced.

Loss of function experiments using antisense morpholino oligonucleotides indicated that the expression of XSMOC-1 is essential for development to proceed through neurulation and subsequent dorsal patterning. In the absence of XSMOC-1, gastrulation and neural induction appeared normal, but embryological development arrested just prior to neurulation (FIG. 6F), in a manner suggestive of the phenotype observed following simultaneous knockdown of chordin, follistatin, and noggin (37). However, these antagonists are expressed during gastrulation in or near the Spemann organizer, and so are likely influencing a set of events distinct in both space and time from those modulated by XSMOC-1.

### EXAMPLE 6

### Experimental Procedures

Isolation of Xenopus SMOC-1: Initial cDNA sequences encoding Xenopus SMOC were obtained following 5′- and 3′-SMART™-RACE (Clontech, CA) amplification using mRNA from stage 59 limbs and degenerate primers designed to sequences conserved between human SMOC1/2 located at the boundary of the follistatin-like and thyroglobulin-like domain 1 (5′-CCACACAYYTGGRYRYRTCTTTGCA-3′) (SEQ ID NO:5) and the extracellular calcium-binding domain (5′-TGGARGCVCTCWCCACHGACATGGT-3′) (SEQ ID NO:6). Full length Xenopus SMOC-1 (Accession number EU287947) was obtained by RT-PCR using stage 59 limb cDNA and the primers 5′-CCTTCATACAAGTCTCACGCCTGA-3′ (SEQ ID NO:7) and 5′-CTTCTTCTGGCCGGCTCTCCTA-3′ (SEQ ID NO:8). PCR products were cloned into pCR®4-TOPO (Invitrogen) and confirmed by sequencing. XSMOC-1 was subsequently subcloned into pCS2 and pcDNA3.

Plasmids and Probes: Zebrafish SMOC-2, obtained from the Zebrafish International Resource Center (clone id CB488) as full-length EST in pSPORT1, was subcloned into pCS2 (provided by David Turner). BMP2, Activin and LM-Smadlwere kind gifts from Gerald Thomsen, Sergei Sokol, and Joan Massagué respectively. Noggin was isolated from stage 10.5 Xenopus cDNA by RT-PCR, and confirmed by sequencing in both directions. Constitutively active chicken BMPR1B was kindly provided by Lee Niswander in the avian retroviral expression vector RCAS BP(A), from which the open reading frame was amplified by PCR using the primers 5′-GTTTTCTGGACAAGATGCCCTT-3′ (SEQ ID NO:9) and 5′-CTCCATCAGAGCTTAATGTCCT-3′ (SEQ ID NO:10). The product was sequenced and subcloned into pCS2. XSox2 (image clone 3398743) and XNot (image clone 8318484) were in pCMVSport6 and pExpress respectively. XMyf5 was isolated by RT-PCR using mRNA from stage 11 Xenopus embryos and was subcloned into PCR-Script™ (Stratagene). Xenopus SMOC-1 antisense morpholino oligonucleotides were as follows: XSMOC-1 MO (5′-GTCATGTTGCCTCTTCTTATACAGG-3′) (SEQ ID NO:11), XSMOC-1 MO 5 base mismatch control (5′-GTgATcTTGCgTCTTgTTATAgAGG-3′) (SEQ ID NO:12), and XSMOC-1 MO2 (5′-CAATCAGGCGTGAGACTTGTATGAA-3′) (SEQ ID NO:13). Each was tagged with fluorescein and purchased from Gene Tools.

Embryo manipulations: Frogs and their embryos were maintained and manipulated using standard methods (12,13). All embryos were staged according to Nieuwkoop and Faber (14) and Keller (15). mRNA injection experiments were performed by standard procedures as described previously (16). Dorsal and ventral blastomeres were identified by size and pigment variations (14). Animal cap explants were cultured in 0.7×Marc's Modified Ringer's (MMR) solution (13) containing 1 mg/ml BSA and 50 mg/ml gentamicin. mRNAs were injected into both blastomeres at the two cell stage or dorsal blastomeres at the four cell stage. For conjugated animal cap assays, animal caps were removed from stage 9 embryos, conjugated immediately, and cultured in 0.7×MMR, 1 mg/ml BSA/50 mg/ml gentamicin until non-injected siblings reached stage 17.

Perturbations of axial patterning were quantified by Dorso-Anterior Index (DAI, (17)). Darkfield images of embryos were photographed with low angle oblique illumination and a Zeiss Stemi-6 dissecting microscope.

Immunoblotting: XSMOC-1 (300 pg) was injected equatorially into each blastomere of Xenopus embryos at the four-cell stage and animal caps, isolated at stage 9, were incubated in 0.7×MMR, 1 mg/ml BSA. 50 mg/ml gentamicin until sibling embryos reached stage 17. Animal caps were extracted on ice in 20 mM Tris pH 7.5, 5 mM EDTA, 2 mM EGTA, 30 mM sodium fluoride, 40 mM β-glycerophosphate, 20 mM sodium pyrophosphate, 1 mM sodium orthovanadate, 1 mM phenylmethyl sulfonyl fluoride, 3 mM benzamidine, 5 mM pepstatin A, 10 mM leupeptin and 0.5% nonidet-p40 in a volume of 10 μl/cap. Supernatants (10 μg/lane) were analyzed by SDS-PAGE using Novex 10% Nu-PAGE gels and the MES buffer system. Immunoblot analysis was performed using the mini-PROTEAN II system (BioRad) and Immobilon™-P PVDF membranes (Millipore). Diphospho-ERK was detected using the rabbit phospho p44/42 MAPK primary antibody (Cell Signaling), goat anti-rabbit HRP-conjugated secondary antibody (Pierce), and SuperSignal® West Femto Maximum Sensitivity Substrate (Pierce).

RT-PCR: Separate pools of embryos or explants from at least two different fertilizations were prepared and analyzed for each condition reported. Total RNA was prepared with Trizol™ and treated with DNA-free™ DNAse removal reagent (Ambion). Reverse transcription (RT) was done using Taqman® RT reagents (Applied Biosystems) as described by the manufacturer, using 1 μg total RNA per reaction; 2% of the cDNA obtained was used in each PCR. Amplification was performed in 10 μl reactions containing 40 mM Tricine-KOH, pH 8.7, 15 mM KOAc, 3.5 mM Mg(OAc)₂, 0.375% bovine albumin, 2.5% Ficoll 400, 5 mM cresol red, 200 μM dNTPs, 0.5 μM each primer, and 0.2 U Advantage® 2 polymerase (Clontech). Each cycle comprised 94° C., 0 seconds; 55° C., 0 seconds; 72° C., 40 seconds; a 1 minute denaturation at 94° C. preceded cycling and a 2 minute extension at 72° C. was included after the final cycle. An Idaho Technologies air thermal cycler was used in all experiments, allowing momentary (setting of ‘0 sec’) dwell times at the annealing and denaturation temperatures to increase amplification specificity. Optimal cycle numbers and annealing temperatures were determined for each primer set. PCR products were separated on 2% agarose gels in TAE buffer, stained with SYBR Green 1™ (Molecular Probes, Eugene, Oreg.), and scanned using a Molecular Dynamics Fluorimager. PCR analysis was performed at least twice for each cDNA to confirm that the amplifications were reproducible. The Xenopus primers for Histone H4, Brachyury, cardiac actin, engrailed, keratin, Krox-20, N-CAM, N-tubulin, and Otx2 are available on the world wide web at xenbase.org, those for Myf-5, Pax6 and XAG-1 are available on the world wide web at hhmi.ucla.edu/derobertis, those for XVent-1 are from Gawantka et al., 1995 and NRP-1 are 5′-GAGTCGCCAGAGACCGAATGGA-3′ (SEQ ID NO:14) and 5′-CATGGCATCATCCACCTTCCCAA-3′ (SEQ ID NO:15).

Hybridization in situ: cRNA probes were produced using MEGAscript T3, T7, or SP6 in vitro transcription kits (Ambion), incorporating digoxigenin. For whole mount hybridization in situ on Xenopus embryos, procedures outlined by Harland were followed (18), with modifications as described (16). For colorimetric detection, signals were developed using alkaline-phosphatase conjugated antibodies to digoxigenin and BM-Purple (Roche). Overstained embryos were embedded in JB-4 resin (Polysciences, Warrington, Pa.) after abbreviated infiltration (3×10 min) and sectioned at 20 microns with a Leica RM2265 rotary microtome.

Histology: Paraffin embedded embryos were sectioned at 7 microns and stained using a modification the Feulgen, light green, orange G method (19). Briefly, deparaffinized sections were incubated overnight at room temperature in fresh Fuelgen stain, rinsed, incubated for 5 minutes in light green (0.2% in 95% ethanol), rinsed and incubated for 30 minutes in orange G (0.2% in 0.2% phosphotungstic acid)

Embryos embedded in JB-4 resin, according to manufacturer's instructions, were sectioned at 3 microns. To accentuate the cement gland and clearly differentiate yolk platelets from other tissues, a modified Van Gieson stain was used. Sections were stained for one hour in 1% Celestine Blue/5% ferric ammonium sulfate, washed in water, stained in 3× Weigert's hematoxylin (3% in 95% ethanol) for 30 seconds, rinsed sequentially with water, 0.37% HCl in 70% ethanol, and 0.07% ammonia in water. The acid alcohol wash was for 2-5 dips, sufficient to remove background Celestine Blue stain; the ammonia water staining was similar, but appearance of light blue background was used as the stopping point. After a 20 minute water wash, the embryos were stained with Van Gieson's solution (20 mL 1% acid fuchsin in water plus 25 mL saturated picric acid) until adequate color balance was achieved (2-5 minutes). Picric acid was from Fluka; all other reagents were from Sigma.

Cell Culture: Mouse 3T3 fibroblasts (1×10⁶) were transiently transfected with 3 μg pcDNA3 or pcDNA3-XSMOC-1 using Nucleofector kit R (Amaxa Biosystems). Following transfection, cells were cultured in DMEM/10% FCS for 18 hours, then incubated in serum-free DMEM for 30 minutes before addition of recombinant human BMP2 (Cell Signaling) for 1 hour. Medium was removed and cells lysates extracted in 6M urea/25 mM Tris/2% SDS containing Halt™ protease and phosphatase inhibitors (Thermo Scientific). BMP2 activity was determined by SDS-PAGE followed by immunoblot analysis of phospho-Smad 1, 5, 8 (the phosphorylation site is conserved among each paralog) and Smad1 (Cell Signaling) using an Odyssey imager and IRDye®800-labeled secondary antibodies (LI-COR Biosciences).

### REFERENCES

1. Vonica, A., and Brivanlou, A. H. (2006) Semin Cell Dev Biol 17, 117-132

2. Chang, S. C., Hoang, B., Thomas, J. T., Vukicevic, S., Luyten, F. P., Ryba, N. J., Kozak, C. A., Reddi, A. H., and Moos, M., Jr. (1994) J Biol Chem 269, 28227-28234

3. Hoang, B., Moos, M., Jr., Vukicevic, S., and Luyten, F. P. (1996) J Biol Chem 271, 26131-26137

4. Vannahme, C., Gosling, S., Paulsson, M., Maurer, P., and Hartmann, U. (2003) Biochem J 373, 805-814

5. Vannahme, C., Smyth, N., Miosge, N., Gosling, S., Frie, C., Paulsson, M., Maurer, P., and Hartmann, U. (2002) J Biol Chem 277, 37977-37986

6. Rocnik, E. F., Liu, P., Sato, K., Walsh, K., and Vaziri, C. (2006) J Biol Chem 281, 22855-22864

7. Raines, E. W., Lane, T. F., Iruela-Arispe, M. L., Ross, R., and Sage, E. H. (1992) Proc Natl Acad Sci USA 89, 1281-1285

8. Kupprion, C., Motamed, K., and Sage, E. H. (1998) J Biol Chem 273, 29635-29640

9. Hasselaar, P., and Sage, E. H. (1992) J Cell Biochem 49, 272-283

10. Francki, A., Bradshaw, A. D., Bassuk, J. A., Howe, C. C., Couser, W. G., and Sage, E. H. (1999) J Biol Chem 274, 32145-32152

11. Liu, P., Lu, J., Cardoso, W. V., and Vaziri, C. (2008) Mol Biol Cell 19, 248-261

12. Gurdon, J. B. (1967). in Methods in Developmental Biology, Crowell, New York

13. Sive, H. L., Grainger, R. M., and Harland, R. M. (2000). in Early Development of Xenopus laevis, Cold Spring Harbor Laboratory Press, Cold Spring Harbor

14. Nieuwkoop, P. D., and Faber, J. (1967) Normal Table of Xenopus Laevis. (Daudin ed., Amsterdam: North Holland

15. Keller, R. (1991) Methods Cell Biol 36, 61-113

16. Moos, M., Jr., Wang, S., and Krinks, M. (1995) Development 121, 4293-4301

17. Kao, K. a. E., R P. (1988) Developmental Biology 127, 64-77

18. Harland, R. M. (1991) Methods Cell Biol 36, 685-695

19. Cooke, J. (1979) J Embryo Exp Morph 51, 165-182

20. Hsu, D. R., Economides, A. N., Wang, X., Eimon, P. M., and Harland, R. M. (1998) Mol Cell 1, 673-683

21. Smith, W. C., and Harland, R. M. (1992) Cell 70, 829-840

22. Heasman, J. (2002) Dev Biol 243, 209-214

23. Clement, J. H., Fettes, P., Knochel, S., Lef, J., and Knochel, W. (1995) Mech Dev 52, 357-370

24. Lin, J., Patel, S. R., Cheng, X., Cho, E., Levitan, I., Ullenbruch, M., Phan, S. H., Park, J. M. and Dressler, G. R. (2005) Nature Med 11, 387-393

25. Zou, H., Wieser, R., Massague, J., and Niswander, L. (1997) Genes Dev 11, 2191-2203

26. Massague, J., Seoane, J., and Wotton, D. (2005) Genes Dev 19, 2783-2810

27. Kretzschmar, M., Doody, J., and Massague, J. (1997) Nature 389, 618-622

28. Pera, E. M., Ikeda, A., Eivers, E., and De Robertis, E. M. (2003) Genes Dev 17, 3023-3028

29. Sapkota, G., Alarcon, C., Spagnoli, F. M., Brivanlou, A. H., and Massague, J. (2007) Mol Cell 25, 441-454

30. Favata, M. F., Horiuchi, K. Y., Manos, E. J., Daulerio, A. J., Stradley, D. A., Feeser, W. S., Van Dyk, D. E., Pitts, W. J., Earl, R. A., Hobbs, F., Copeland, R. A., Magolda, R. L., Scherle, P. A., and Trzaskos, J. M. (1998) J. Biol. Chem. 273, 18623-18632

31. Hoodless, P. A., Haerry, T., Abdollah, S., Stapleton, M., O'Connor, M. B., Attisano, L., and Wrana, J. L. (1996) Cell 85, 489-500

32. Dudley, A., Lyons, K M, Robertson, E J. (1995) Genes and Development 9, 2795-2807

33. Wang, S., Krinks, M., Kleinwaks, L., and Moos, M., Jr. (1997) Genes Funct 1, 259-271

34. Chang, C., and Hemmati-Brivanlou, A. (1999) Development 126, 3347-3357

35. Hemmati-Brivanlou, A., and Thomsen, G. H. (1995) Dev Genet 17, 78-89

36. Wieser, R., Wrana, J. L., and Massague, J. (1995) EMBO J 14, 2199-2208

37. Khoka, M. K., Yeh, J., Grammer, T. C., and Harland, R. M. (2005) Developmental Cell 8, 401-411

38. Harland, R., and Gerhart, J. (1997) Annu Rev Cell Dev Biol 13, 611-667

39. Hemmati-Brivanlou, A., and Melton, D. (1997) Cell 88, 13-17

40. Weinstein, D. a. H.-B., A. (1999) Annual Review of Cell and Developmental Biology 15, 411-433

41. Wallingford, J. B., Fraser, Scott E., Harland, Richard M. (2002) Developmental Cell 2, 695-706

42. Yamada, K. M., and Even-Ram, S. (2002) Nat Cell Biol 4, E75-76

43. Lallier, T. E., and DeSimone, D. W. (2000) Dev Biol 225, 135-150

44. Lallier, T. E., Whittaker, C. A., and DeSimone, D. W. (1996) Development 122, 2539-2554

45. Paulsson, M. (1992) Crit Rev Biochem Mol Biol 27, 93-127

46. Barker, T. H., Baneyx, G., Cardo-Vila, M., Workman, G. A., Weaver, M., Menon, P. M., Dedhar, S., Rempel, S. A., Arap, W., Pasqualini, R., Vogel, V., and Sage, E. H. (2005) J Biol Chem 280, 36483-36493

47. Giancotti, F. G., and Tarone, G. (2003) Ann Rev Cell Dev Biol 19, 173-206

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method for modulating growth and differentiation of bone and cartilage in a patient comprising administering to the patient an effective amount of a secreted modular calcium binding protein;
a nucleic acid encoding a secreted modular calcium binding protein;
a vector comprising a nucleic acid encoding a secreted modular calcium binding protein;
or
a host cell expressing a secreted modular calcium binding protein.

2. The method of claim 1 wherein the secreted modular calcium binding protein is human secreted modular calcium binding protein-1, human secreted modular calcium binding protein-2, or Xenopus laevis secreted modular calcium binding protein.

3. The method of claim 2 wherein the human secreted modular calcium binding protein-1 comprises the amino acid sequence of SEQ ID NO:2 or a biologically active fragment or conservatively modified variant thereof.

4-5. (canceled)

6. The method of claim 5 claim 2 wherein the Xenopus laevis secreted modular calcium binding protein comprises the amino acid sequence of SEQ ID NO:4 or a biologically active fragment or conservatively modified variant thereof.

7. (canceled)

8. The method of claim 1 wherein the nucleic acid encoding a secreted modular calcium binding protein encodes human secreted modular calcium binding protein-1, human secreted modular calcium binding protein-2, or Xenopus laevis secreted modular calcium binding protein.

9. The method of claim 8 wherein the nucleic acid encoding human secreted modular calcium binding protein-1 comprises a nucleotide sequence that is at least 95% identical to the nucleotide sequence of SEQ ID NO:1.

10-11. (canceled)

12. The method of claim 11 claim 8 wherein the nucleic acid encoding Xenopus laevis secreted modular calcium binding protein comprises a nucleotide sequence that is at least 95% identical to the nucleotide sequence of SEQ ID NO:3.

13. (canceled)

14. A method for treating a musculoskeletal disorder comprising administering to a patient suffering from such a disorder a therapeutically effective amount of a secreted modular calcium binding protein;
a nucleic acid encoding a secreted modular calcium binding protein;
a vector comprising a nucleic acid encoding a secreted modular calcium binding protein;
or
a host cell expressing a secreted modular calcium binding protein.

15. The method of claim 14 wherein the musculoskeletal disorder is a joint disorder.

16. The method of claim 15 wherein the joint disorder is spondylarthropathic disease.

17. The method of claim 14 wherein the secreted modular calcium binding protein is human secreted modular calcium binding protein-1, human secreted modular calcium binding protein-2, or Xenopus laevis secreted modular calcium binding protein.

18. The method of claim 17 wherein the human secreted modular calcium binding protein-1 comprises the amino acid sequence of SEQ ID NO:2 or a biologically active fragment or conservatively modified variant thereof.

19-20. (canceled)

21. The method of claim 17 wherein the Xenopus laevis secreted modular calcium binding protein comprises the amino acid sequence of SEQ ID NO:4 or a biologically active fragment or conservatively modified variant thereof

22. (canceled)

23. The method of claim 14 wherein the nucleic acid encoding a secreted modular calcium binding protein encodes human secreted modular calcium binding protein-1, human secreted modular calcium binding protein-2, or Xenopus laevis secreted modular calcium binding protein.

24. The method of claim 23 wherein the nucleic acid encoding human secreted modular calcium binding protein-1 comprises a nucleotide sequence that is at least 95% identical to the nucleotide sequence of SEQ ID NO:1.

25-26. (canceled)

27. The method of claim 23 wherein the nucleic acid encoding Xenopus laevis secreted modular calcium binding protein comprises a nucleotide sequence that is at least 95% identical to the nucleotide sequence of SEQ ID NO:3.

28. (canceled)

29. A method for modulating bone morphogenetic protein activity comprising activating an extracellular signal-regulated mitogen-activated protein kinase with a secreted modular calcium binding protein.

30. The method of claim 29 wherein the secreted modular calcium binding protein is human secreted modular calcium binding protein-1, human secreted modular calcium binding protein-2, or Xenopus laevis secreted modular calcium binding protein.

31. The method of claim 30 wherein the human secreted modular calcium binding protein-1 comprises the amino acid sequence of SEQ ID NO:2 or a biologically active fragment or conservatively modified variant thereof.

32-33. (canceled)

34. The method of claim 30 wherein the Xenopus laevis secreted modular calcium binding protein comprises the amino acid sequence of SEQ ID NO:4 or a biologically active fragment or conservatively modified variant thereof

35. (canceled)


SEQUENCE LISTING (18 sequences)

SEQ ID NO: 1 - DNA, 1305 bp, Homo sapiens
atgctgcccgcgcgctgcgcccgcctgctcacgccccacttgctgctggtgttggtgcagctgtcccctgctcgcggccaccgcaccacaggccccaggtttctaataagtgaccgtgacccacagtgcaacctccactgctccaggactcaacccaaacccatctgtgcctctgatggcaggtcctacgagtccatgtgtgagtaccagcgagccaagtgccgagacccgaccctgggcgtggtgcatcgaggtagatgcaaagatgctggccagagcaagtgtcgcctggagcgggctcaagccctggagcaagccaagaagcctcaggaagctgtgtttgtcccagagtgtggcgaggatggctcctttacccaggtgcagtgccatacttacactgggtactgctggtgtgtcaccccggatgggaagcccatcagtggctcttctgtgcagaataaaactcctgtatgttcaggttcagtcaccgacaagcccttgagccagggtaactcaggaaggaaagatgacgggtctaagccgacacccacgatggagacccagccggtgttcgatggagatgaaatcacagccccaactctatggattaaacacttggtgatcaaggactccaaactgaacaacaccaacataagaaattcagagaaagtctattcgtgtgaccaggagaggcagagtgccctggaagaggcccagcagaatccccgtgagggtattgtcatccctgaatgtgcccctgggggactctataagccagtgcaatgccaccagtccactggctactgctggtgtgtgctggtggacacagggcgcccgctgcctgggacctccacacgctacgtgatgcccagttgtgagagcgacgccagggccaagactacagaggcggatgaccccttcaaggacagggagctaccaggctgtccagaagggaagaaaatggagtttatcaccagcctactggatgctctcaccactgacatggttcaggccattaactcagcagcgcccactggaggtgggaggttctcagagccagaccccagccacaccctggaggagcgggtagtgcactggtatttcagccagctggacagcaatagcagcaacgacattaacaagcgggagatgaagcccttcaagcgctacgtgaagaagaaagccaagcccaagaaatgtgcccggcgtttcaccgactactgtgacctgaacaaagacaaggtcatttcactgcctgagctgaagggctgcctgggtgttagcaaagaaggacgcctcgtctaa

SEQ ID NO: 2 - protein, 434 aa, Homo sapiens
MLPARCARLLTPHLLLVLVQLSPARGHRTTGPRFLISDRDPQCNLHCSRTQPKPICASDGRSYESMCEYQRAKCRDPTLGVVHRGRCKDAGQSKCRLERAQALEQAKKPQEAVFVPECGEDGSFTQVQCHTYTGYCWCVTPDGKPISGSSVQNKTPVCSGSVTDKPLSQGNSGRKDDGSKPTPTMETQPVFDGDEITAPTLWIKHLVIKDSKLNNTNIRNSEKVYSCDQERQSALEEAQQNPREGIVIPECAPGGLYKPVQCHQSTGYCWCVLVDTGRPLPGTSTRYVMPSCESDARAKTTEADDPFKDRELPGCPEGKKMEFITSLLDALTTDMVQAINSAAPTGGGRFSEPDPSHTLEERVVHWYFSQLDSNSSNDINKREMKPFKRYVKKKAKPKKCARRFTDYCDLNKDKVISLPELKGCLGVSKEGRLV

SEQ ID NO: 3 - DNA, 1392 bp, Xenopus laevis
atgaccccaagactagtgatcgcactttttgtaacttgctactttgtccttctgcatctgcctcagtgtttcggccaaagagcgactggccccaggtttttaataagtgatcgggacagagaccctcagtgcaatcctcactgcaccaggccacaacataaaccagtatgtgcttcagatggtcggacctatgaatctatgtgcgattatcaacgagccaagtgcaaggactcttcccttagtgtgacccaacgtggaaggtgcaaagatgctggtcagagcaagtgtcgtctggaaaggactcaagcgcttgaacaggcgaagaagccccaggaagctgtgtttatacctgaatgcaatgaagatggatcatttactcaagtgcaatgtcatacgttcacaggatactgctggtgcgtcacccctgatggcaaacctgtaagcggctcttcagtgcagaacaaaacacctgtatgttcaggttctgtaactgagaagccctcaagtcagggcaattccggcaggagagggggaagaggccctcacccaatacttaggggtgaaattcagagttcggatcatccatcgcaaaacgtagggggtggggtcatagacatcaccgctcctacgctctggataaagcaccttgttatcaaagatgccaaactaaacagctcaagcgttaaacacccagagaaggcaaattcctgcgatcaggagcgacaaagtgctctggaggaagccaaactgaacccaagagagggaattgtgattccagaatgtgccccaggcggcctttacaaacctgtgcagtgccaccagtccactggatattgttggtgtgtcttagtagacactggtcgtccattgcctggaacatctacacgttatgaaactcctgtgtgcgaaagtgatgccagatggaaaaatacggacgctgaagacccatttaaagacagagaattagcaggttgtccagaaggaaagaagctggaattcatcaccagtttattggacgctctcaccacggacatggtgcaagccatcaactcagcagcgccggccgcaggcgggagattctcagagccagatcccaatcacacactggaggaaagagtggtcctttggtacttcagccagttagacagcaacgccagtgaggacataaataagaaggagatgaagcccttcaaaagatacgtaaaaaagaaagccaaacccaagaaatgtgcccggcgtttcactgactactgtgatcttaataaggacaaatccatttcatttccagagcttaaaggctgtctaggagtccgcaaggaaccaggtgcaaatgctggaagtttccctcctggaaagcggcccggctctaaccccttcagtcgccttgcgtag

SEQ ID NO: 4 - protein, 463 aa, Xenopus laevis
MTPRLVIALFVTCYFVLLHLPQCFGQRATGPRFLISDRDRDPQCNPHCTRPQHKPVCASDGRTYESMCDYQRAKCKDSSLSVTQRGRCKDAGQSKCRLERTQALEQAKKPQEAVFIPECNEDGSFTQVQCHTFTGYCWCVTPDGKPVSGSSVQNKTPVCSGSVTEKPSSQGNSGRRGGRGPHPILRGEIQSSDHPSQNVGGGVIDITAPTLWIKHLVIKDAKLNSSSVKHPEKANSCDQERQSALEEAKLNPREGIVIPECAPGGLYKPVQCHQSTGYCWCVLVDTGRPLPGTSTRYETPVCESDARWKNTDAEDPFKDRELAGCPEGKKLEFITSLLDALTTDMVQAINSAAPAAGGRFSEPDPNHTLEERVVLWYFSQLDSNASEDINKKEMKPFKRYVKKKAKPKKCARRFTDYCDLNKDKSISFPELKGCLGVRKEPGANAGSFPPGKRPGSNPFSRLA

SEQ ID NO: 5 - DNA, 25 bp, Artificial Sequence
  Synthetic primer
ccacacayytggryryrtctttgca

SEQ ID NO: 6 - DNA, 25 bp, Artificial Sequence
  Synthetic primer
tggargcvctcwccachgacatggt

SEQ ID NO: 7 - DNA, 24 bp, Artificial Sequence
  Synthetic primer
ccttcatacaagtctcacgcctga

SEQ ID NO: 8 - DNA, 22 bp, Artificial Sequence
  Synthetic primer
cttcttctggccggctctccta

SEQ ID NO: 9 - DNA, 22 bp, Artificial Sequence
  Synthetic primer
gttttctggacaagatgccctt

SEQ ID NO: 10 - DNA, 22 bp, Artificial Sequence
  Synthetic primer
ctccatcagagcttaatgtcct

SEQ ID NO: 11 - DNA, 25 bp, Artificial Sequence
  Synthetic primer
gtcatgttgcctcttcttatacagg

SEQ ID NO: 12 - DNA, 25 bp, Artificial Sequence
  Synthetic primer
gtgatcttgcgtcttgttatagagg

SEQ ID NO: 13 - DNA, 25 bp, Artificial Sequence
  Synthetic primer
caatcaggcgtgagacttgtatgaa

SEQ ID NO: 14 - DNA, 22 bp, Artificial Sequence
  Synthetic primer
gagtcgccagagaccgaatgga

SEQ ID NO: 15 - DNA, 23 bp, Artificial Sequence
  Synthetic primer
catggcatcatccaccttcccaa

SEQ ID NO: 16 - protein, 435 aa, Homo Sapiens
  misc_feature (435)..(435): Xaa can be any naturally occurring amino acid
MLPARCARLLTPHLLLVLVQLSPARGHRTTGPRFLISDRDPQCNLHCSRTQPKPICASDGRSYESMCEYQRAKCRDPTLGVVHRGRCKDAGQSKCRLERAQALEQAKKPQEAVFVPECGEDGSFTQVQCHTYTGYCWCVTPDGKPISGSSVQNKTPVCSGSVTDKPLSQGNSGRKDDGSKPTPTMETQPVFDGDEITAPTLWIKHLVIKDSKLNNTNIRNSEKVYSCDQERQSALEEAQQNPREGIVIPECAPGGLYKPVQCHQSTGYCWCVLVDTGRPLPGTSTRYVMPSCESDARAKTTEADDPFKDRELPGCPEGKKMEFITSLLDALTTDMVQAINSAAPTGGGRFSEPDPSHTLEERVVHWYFSQLDSNSSNDINKREMKPFKRYVKKKAKPKKCARRFTDYCDLNKDKVISLPELKGCLGVSKEGRLVX

SEQ ID NO: 17 - protein, 461 aa, Xenopus sp.
MTPRLVIALFVTCYFVLLHLPQCFGQRATGPRFLISDRDRDPQCNPHCTRPQHKPVCASDGRTYESMCDYQRAKCKDSSLSVTQRGRCKDAGQSKCRLERTQALEQAKKPQEAVFIPECNEDGSFTQVQCHTFTGYCWCVTPDGKPVSGSSVQNKTPVCSGSVTEKPSSQGNSGRRGRRGPHPILRGEIQSSDHPSQNVGGGVIDITAPTLWIKHLVIKDAKLNSSSVKHPEKANSCDQERQSALEEAKLNPREGIVIPECPGGLYKPVCHQSTGYCWCVLVDTGRPLPGTSTRYETPVCESDARWKNTDAEDPFKDRELAGCPEGKKLEFITSLLDALTTDMVQAINSAAPAAGGRFSEPDPNHTLEERVVLWYFSQLDSNASEDMNKKEMKPFKRYVKKKAKPKKCARRFTDYCDLNKDKSISFPELKGCLGVRKEPGANAGSFPPGKRPGSNPFSRLA

SEQ ID NO: 18 - protein, 333 aa, Artificial Sequence
  Synthetic construct
MPLVLLGRTGPRFLISDRDPQCNHCRQKPCASDGRYESMCYQRAKCDLVRGRCKDAGQSKCRLERQALEQAKKPQEAVFPECEDGSFTQVQCHTTGYCWCVTPDGKPSGSSVQNKTPVCSGSVTKPSQGNSGRGPPGITAPTLWIKHLVIKDKLNEKSCDQERQSALEEANPREGIVIPECGKPVQSTGYCWCVLVDTGRPLPGTSTRYPCESDARKTADPFKDRELGCPEGKKEFITSLLDALTTDMVQAINSAAPGGRFSEPDPHTLEERVVWYFSQLDSNSDNKEMKPFKRYVKKKAKPKKCARRFTDYCDLNKDKISPELKGCLGVKEG